(19) United States
(12) Patent Application Publication
(10) Pub. No.: US 2024/0277819 A1
(43) Pub. Date: Aug. 22, 2024

(54) COMPOSITIONS AND METHODS FOR DEBULKING VIRUS OR MICROORGANISM LOAD IN THE ORAL CAVITY

(71) Applicant: THE TRUSTEES OF THE UNIVERSITY OF PENNSYLVANIA, Philadelphia, PA (US)

(72) Inventors: Henry DANIELL, Media, PA (US); Robert P. RICCIARDI, Kennett Square, PA (US)

(73) Assignee: THE TRUSTEES OF THE UNIVERSITY OF PENNSYLVANIA, Philadelphia, PA (US)

(21) Appl. No.: 18/560,153

(22) PCT Filed: May 11, 2022
(86) PCT No.: PCT/US2022/028801

§ 371 Date: Nov. 10, 2023

Related U.S. Application Data
(60) Provisional application No. 63/187,924, filed on May 12, 2021.

(51) Int. Cl. A61K 38/48; A61K 9/68; A61K 31/728; A61K 38/16; A61K 47/02; A61K 47/12; A61K 47/26; A61P 31/14; A61P 31/16

(52) U.S. Cl. CPC A61K 38/48; A61K 9/0058; A61K 31/728; A61K 38/168; A61K 47/02; A61K 47/12; A61K 47/26; A61P 31/14; A61P 31/16; C12Y 304/17023

(57) ABSTRACT

Compositions and methods for reducing viral load in the oral cavity, particularly Coronavirus such as SARS-CoV-2 and Influenza viral loads are disclosed. Also disclosed are compositions and methods for reducing bacterial or fungal loads in the oral cavity. The compositions comprise a trapping molecule having binding affinity for a protein, glucan, or other molecule on the surface of a virus or microorganism. A carrier appropriate for oral administration, in the form of a chewing gum, long-acting lozenge, or tablet, is further disclosed, to enable easy administration before or after exposure to the infective agent.

### CROSS REFERENCE TO RELATED APPLICATION

This application claims the benefit of U.S. Provisional Application No. 63/187,924 filed May 12, 2021, the entire contents being incorporated herein by reference as though set forth in full.

### STATEMENT OF GRANT SUPPORT

This invention was made with government support under R01HL107904 awarded by the National Institutes of Health. The government has rights in the invention.

### FIELD OF THE INVENTION

The present invention relates to the fields of virus infections and anti-viral compositions. More specifically, the invention provides compositions and methods which effectively reduce the viral load in the oral cavity, thereby reducing viral transmission, particularly of SARS-CoV-2, influenza and other transmissible viruses. Also provided are compositions and methods for reducing bacterial and fungal loads in the oral cavity.

### BACKGROUND OF THE INVENTION

Several publications and patent documents are cited throughout the specification in order to describe the state of the art to which this invention pertains. Each of these citations is incorporated by reference herein as though set forth in full.

SARS-CoV-2 transmission occurs through both droplet and aerosol transmission and is linked in large part with indoor exposure to infected individuals, symptomatic or asymptomatic. Controlling transmission has involved reduction of concentrations of indoor aerosols largely through masking and physical distancing. In public buildings (classrooms, retail shops, restaurants, gyms, churches, etc.), 4 to 6 air changes per hour through outdoor air ventilation, recirculated air through a minimum efficiency rating value 13 (MERV 13) rating or passage of air through HEPA filters are recommended (Allen. 2021). While larger particles (>100 μm) could settle down by gravitational forces, most people emit >100 times smaller aerosols (<5 μm) during talking, breathing or coughing.

A high SARS-CoV-2 viral load is often detected in saliva. Highly contagious airborne droplets are the major cause of transmission in respiratory viruses like influenza, measles, and SARS-CoV2. Human Papillomavirus, Herpes Simplex virus type 1. Epstein-Barr virus and Kaposi's sarcoma-associated herpesvirus are orally transmitted and their replication in the oral epithelium is well known. In SARS-CoV-2 with saliva average load of 7×10⁶ copies of RNA virus per ml, an oral fluid droplet of 50 μm² could contain at least one virion. High SARS-CoV-2 viral loads are detected in saliva of both asymptomatic and symptomatic COVID-19 patients. In fact, salivary viral burden correlates with the severity of COVID-19 symptoms including the loss of taste and smell, and the virus replicates in salivary glands and oral mucous membranes. Thus, the oral mucous membranes and saliva appear to be a high-risk route for SARS-CoV-2 transmission.

Currently, there are no compositions and methods which specifically and effectively debulk viruses from the oral cavity and throat. It is an object of the invention to address this need.

### SUMMARY OF THE INVENTION

In accordance with the present invention, a method for debulking viral load in the oral cavity is disclosed. An exemplary method comprises orally administering to the subject a therapeutically effective amount of a composition comprising a carrier and a trapping molecule which binds a protein or glucan (simple or complex) on the surface of a virus, wherein binding of the surface protein or glucan to the trapping molecule traps the virus within said carrier and reduces viral load in the oral cavity. In certain embodiments, reduction of viral load in the oral cavity of said subject reduces transmission of the virus. In certain embodiments, the administering takes place before the subject is exposed to the virus (e.g., for use a personal protection equipment (PPE). In other embodiments, the administering takes place after the subject is exposed to the virus. Preferably, the administering reduces viral load, reduces recovery time for, eliminates, or minimizes at least one complication from the viral infection and, or reduces viral transmission. The virus includes any virus that resides in the oral cavity. Such viruses include, without limitation Corona viruses, Herpes viruses, Papilloma viruses, Influenza Viruses, Epstein Barr virus, cytomegaloviruses, Hepatitis C virus, Zika virus and other viruses which use the salivary gland as a reservoir.

In one aspect of the invention, the virus comprises a Coronavirus, e.g., at least one of an Alphacoronavirus, a Betacoronavirus, a Gammacoronavirus, and a Deltacoronavirus. In other aspects, the Coronavirus comprises at least one of MERS-CoV, SARS-CoV, and SARS-CoV-2. In particularly preferred embodiments, the virus is SARS-CoV-2.

In another preferred embodiment, the virus is SARS-CoV-2, the trapping molecule is ACE2 or CTB-ACE2 which binds the spike protein on the viral surface, and traps said virus in said carrier, thereby reducing viral load in the oral cavity.

In another embodiment, the virus is an Alpha influenza virus and comprises at least one of Influenza A virus, Influenza B virus, and Influenza C virus. In this embodiment, the trapping molecule can be an influenza virus A (IVA) blocking peptide which binds portions of influenza hemagglutinin (HA) and neuraminidase (NA) proteins on said influenza virus which traps influenza viral particles in said carrier. In certain embodiments, the IVA blocking peptide comprises virus binding portions of HA and or neuraminidase proteins.

The carrier can be a chewing gum, long-acting lozenge, or a tablet. In preferred embodiments, the carrier is chewing gum.

The invention also provides a composition useful in the methods disclosed above. An exemplary composition comprises a carrier comprising an effective amount of a trapping molecule having binding affinity for a surface protein or glucan (simple or complex) of a virus residing in the oral cavity, which may or may not be transmissible via aerosolization, wherein the carrier is suitable for oral administration. The carrier can be a chewing gum, but can also be a long-acting lozenge, or an oral tablet. In preferred embodiments, the carrier is chewing gum. In particularly preferred embodiments of the composition, the virus to be treated is SARS-CoV-2, said trapping molecule is ACE2 or CTB-ACE2 and the carrier is chewing gum.

In another aspect of the composition, the virus is Influenza A virus, said trapping molecule is an IVA blocking peptide and said carrier is chewing gum. In this aspect the IVA blocking peptide can comprise virus binding portions of HA and or neuraminidase proteins.

In another aspect of the composition, the virus is Influenza A virus, SARS-CoV02, Herpes virus or papilloma virus, said trapping molecule is FRIL isolated from lablab bean powder, and said carrier is chewing gum.

In accordance with yet another embodiment, a method for debulking microorganisms such as bacteria or fungus, in the oral cavity is disclosed. An exemplary method comprises orally administering to the subject a therapeutically effective amount of a composition comprising a carrier and a trapping molecule which binds a protein on the surface of the microorganism, wherein binding of the surface protein to the trapping molecule traps the microorganism within said carrier and microorganism load in the oral cavity. In certain embodiments, reduction of microbial load in the oral cavity of said subject reduces transmission of the microorganism.

The invention also provides a composition useful in the method disclosed above. An exemplary composition comprises a carrier comprising an effective amount of a trapping molecule having binding affinity for a surface protein of a microorganism residing in the oral cavity, which may or may not be transmissible via aerosolization, wherein the carrier is suitable for oral administration. The carrier can be a chewing gum, but can also be a long-acting lozenge, or an oral tablet. In certain embodiments, the microorganism is a bacteria, such as Streptococcus pyogenes and the trapping molecule is an antimicrobial peptide. Suitable antimicrobial peptides for this purpose include, without limitation, protegrin, retrocyclin defensins, PGLA (frog skin), cecropins, apidaecins, melittin, MSI-99, bombinin, magainin, boceprevir, and telaprevir.

In accordance with yet another embodiment, a method for debulking fungal organisms such as C. albicans, in the oral cavity is disclosed. An exemplary method comprises orally administering to the subject a therapeutically effective amount of a composition comprising a carrier and an enzyme such as lipase. Lipids are important membrane components for fungal morphogenesis and hypal elongation (Rella et al 2016). In certain embodiments, reduction of fungal load in the oral cavity of said subject reduces transmission of the fungus.

The invention also provides a composition useful in the method disclosed above. An exemplary composition comprises a carrier and an enzyme such as lipase, wherein the carrier is suitable for oral administration. The carrier can be a chewing gum, but can also be a long-acting lozenge, or an oral tablet. In certain embodiments, the microorganism is a fungus, such as Candida albicans and the trapping molecule is lipase.

### BRIEF DESCRIPTION OF THE DRAWINGS

FIG. 1: Fresh weight per plant (g) and ACE2 expression (mg/g dry weight) at different ages between Aerofarms and Fraunhofer. The ACE2 expression was quantified based on total protein of homogenate (μg/μl) and converted to mg/g of plant dried weight (mg/g DW). The values are mean±SD (n=3). The percentage of ACE2 protein content in total leaf protein is presented on top of each column in Blue. Total leaf protein content (mg/g DW) of CTB-ACE2 plant material is presented on top of each column in red. The values are the mean±SD (n=8). Total fresh biomass (Kg) harvested at different ages is presented on to of each column in black. Plant ages are relative to the date of germination. While growth conditions were different, biomass yield per plant was similar on 80-90 days but declined as plants grew older. However, the most dramatic difference was in the expression level of CTB-ACE2; while the maximum expression for plants grown at Aerofarms was <2 mg CTB-ACE2/g dry weight, the expression level at Fraunhofer USA ranged from 15-22 mg/g dry weight.

FIGS. 2A-2C: Neutralization of SARS-CoV-2 Spike-pseudotyped lentivirus. Vero cells were infected with luciferase-expressing Spike-pseudotyped lentiviral particles. (FIG. 2A) Raw luminescence values. (FIG. 2B) Relative infectivity was determined by the comparison of the luminescence of ACE2-gum to corresponding placebo. Values are shown as mean±SD for n=6 for gum and n=3-4 for virus-only, infected cells or non-virus control. Shown are mean relative infectivity values±SD for n=6 for gum treatments (p<0.0001 for all treatments as determined by two-tailed test) and n=3-4 for virus-only, infected cells or uninfected cells. (FIG. 2C) SARS-CoV-2 spike glycoprotein pseudotyped viruses expressing luciferase were incubated with ACE2 gum at the indicated concentration for 90 minutes at room temperature. Following centrifugation, virus-containing supernatant was incubated with ACE2-expressing CHO cells for 72 hrs, and viral infectivity was measured via luciferase. Data are representative of three independent experiments with N=2-3 samples. Bars represent the condition mean, symbols represent the average of duplicate or triplicate assays, error bars represent the SEM. p<0.01 **p<0.001***, p<0.0001**** by Kruskal-Walis ANOVA.

FIG. 3: Impact of ACE2 on entry of VSV-spike particles: Relative inhibition of VSV-S particle entry after incubation with CTB-ACE2 and ACE2 gum powder was calculated to be statistically significant in all treatment groups, as compared to the untreated VSV-S control. The graph in Figure ** is representative of 2 replicates in 2 independent experiments. (student's t-test, p<0.05) *=p<0.05, **=p<0.01

FIGS. 4A-4B: Neutralization of SARS-CoV-2 in NP swab samples. (FIG. 4A) Images from clinical NP swab samples treated by different conditions. All images share the scale bar. (FIG. 4B). Relative inhibition was determined by the comparison of the number of bubbles for gum treated samples to untreated samples.

FIGS. 5A-5D: Kinetic reading of ACE-2 in saliva and plant derived full length ACE2. FIGS. 5A and 5B: ACE-2 activity determined by cleavage of fluorogenic Mca-APK (Dnp) substrate in 10 control and 10 COVID (red) samples. ΔRFU was calculated by subtracting data of timepoint 0 minutes from data of timepoint 90 minutes. FIG. 5C: ACE-2 enzyme activity presented as enzyme units (mU/mg). Data were analyzed using the Student's 1-test; **: p<0.0024. FIG. 5D: Interaction of full length CTB-ACE2 with or without recombinant SARS-CoV-2 spike proteins. ACE2 activity was measured using 20 μg of CTB-ACE2 protein extracts by cleavage of fluorogenic Mca-APK (Dnp) substrate in the presences and absence of 10 μg spike proteins (SARS-CoV-2 RBD, and SARS-CoV-2 S1-S2). NC, negative control (substrate and buffer only).

FIGS. 6A-6C: Reduction of COVID-19 copies detected by ddPCR in chewing gum incubation: COVID-19 positive Saliva samples were incubated with ACE2 powder gum. (FIG. 6A) N1 target is specific to SARS-CoV-2 and (FIG. 6B) RP target is non-specific. N1 copy number is highly reduced by ACE2 chewing gum. (FIG. 6C) A graph quantifying this data.

FIGS. 7A-7B: FIG. 1: Blocking and neutralization mechanisms of plant-based SARS-CoV-2 viral trap proteins-CTB-ACE2 and FRIL (FIG. 7A) The pentameric insoluble microparticles of the lettuce based CTB-ACE2 in the chewing gum tablet effectively binds to the viral spike proteins and sediments the SARS-CoV-2 virus thus blocking the viral entry into human cells. (FIG. 7B) The homotetramer, FRIL a plant-based lectin (Lablab bean) has strong anti-SARS-CoV-2 and anti-influenza activity by binding to complex-type-N glycans present on the viral surface glycoproteins forming aggregates and by sequestering the virions at the late endosomal stage preventing entry into the nucleus.

FIG. 8: Schematic diagram of the process for CTB-ACE2 chewing gum—from lab to the clinic. The chronological sequence of steps in the chewing gum manufacturing process from creation and characterization of plants expressing CTB-ACE2, production and characterization of the chewing gum and steps towards clinical development. Steps #1 to Step #11 are described in detail in Daniell et al. 2020 and Daniell et al. 2021). Step #8 is described by Per Os Biosciences in the U.S. Pat. No. 9,744,128 patent.

FIGS. 9A-9B: CTB-ACE2 gum tablet release and total dose quantitation (FIGS. 9A, 9B) Western blot analysis for CTB-ACE2 immediate release without sonication. Data presented as mean±SD; n=4. (FIGS. 9C, 9D) Western blot analysis for CTB-ACE2 total dose quantitation. Data presented as means±SD; n=4.

FIGS. 10A-10B: Neutralization of SARS-CoV-2 Omicron variant in NP swab samples with FRIL (FIG. 10A) Images from clinical NP/OP swab samples of patients #620 and #613 treated with FRIL bean powder (20, 40, 100 μg FRIL protein in 5, 10 and 25 mg of bean powder respectively). (FIG. 10B) Number of bubbles are quantified. All images share the same scale bar. Data were analyzed by One-way ANOVA test. There is a significant difference in the microbubble count between the Untreated vs FRIL bean powder at all concentrations. (***p-value<0.0001).

FIGS. 11A-11D: Neutralization of SARS-CoV-2 Delta and Omicron variants in NP swab samples with CTB-ACE2 gum (FIG. 11A) Images from clinical NP/OP swab samples of patients #614, #615 treated with ACE2 gum (0.46 or 0.92 μg CTB-ACE2 protein in 25 and 50 mg of gum respectively) (FIG. 11B) Number of bubbles are quantified. Data were analyzed by One-way ANOVA test. There is a significant difference in the microbubble count between the Untreated Omicron strain vs ACE2 gum at both concentrations. (***p-value=0.0001). All images share the same scale bar. (FIG. 11C) Images from clinical NP/OP swab samples of patients #151. #153 and #155 treated with ACE2 gum (0.46 or 0.92 μg CTB-ACE2 protein in 25 and 50 mg of gum respectively). (FIG. 11D) Number of bubbles are quantified. Data were analyzed by One-way ANOVA test. There is a significant difference in the microbubble count between the Untreated Delta strain vs ACE2 gum at both concentrations. (***p-value=0.0009)

FIG. 12: ACE2 chewing gum trapping effect efficacy assessed through diagnosis with RAPID. Normalized charge-transfer resistance values for the electrochemical impedance spectroscopy measurements of 20 SARS-CoV-2 clinical samples (left y-axis) before and after exposure to 20 mg of ACE2 chewing gum. RNA copies μL⁻¹ from qPCR of infected samples prior to exposure to the ACE2 chewing gum (right y-axis). Numbers below samples are patient IDs. Please refer to table 1 for other details. Sample ID 593 was not genotyped. Samples 595-609 are Delta and 613-631 are Omicron SARS-CoV-2 strain are based on whole genome sequencing or S gene target failure or date of collection. RNA extraction was from 140 μl patient sample (1/50 μl shown) and RAPID was performed in 100 μl patient sample (10 μl Rct value shown).

FIGS. 13A-13F: FRIL Plaque Reduction Neutralization Assay FRIL plaque reduction neutralization assay with H1N1 (A/California/7/2009-X181), H3N2 (A/Singapore/INFMH-16/0019/2016), and HCoV-OC43 virus. The viruses were pre-incubated with increasing amounts of purified FRIL protein or soluble extract of lablab bean powder at 37° C. for 1 hr in 100 μl. The pretreated viruses were then added onto cells for the plaque reduction assay and plaque numbers were quantified at 28 hr post infection for the two Flu viruses and 5 days post infection for HCoV-OC43 virus. The data represents means±SD of plaque numbers from two independent experiments in duplicate. Since 4 mg of Purified FRIL is equivalent 1 mg of FRIL Bean Powder, a greater input weight of FRIL Bean Powder (mg), was required to attain a comparative dose response curve to Purified FRIL (mg). For convenience of comparison, we expressed the 50% weight inhibition for each dose response curve in nanograms (ng) for FRIL protein.

FIG. 14: FRIL Plaque Reduction Neutralization Assay FRIL inhibition of Vero under infective dose of H1N1 (A/California/7/2009/X181) and H3N2 (A/Singapore/INFMB-16/0019/2016).

FIGS. 15A-15B: FRIL entrapped Influenza Virus by Aggregation. (FIG. 15A) Negative-stain EM images of H1N1 (A/California/7/2009-X181) cell culture virions alone (left panels) and aggregated virus particles with FRIL after incubating with 10 μg/mL and 150 μg/mL FRIL (right panels). Data are representative of 2 independent experiments. (FIG. 15B) Negative-stain EM images of sucrose-gradient purified X181 virions alone (left panels) and aggregated X181 virus particles after mixing with 150 μg/mL FRIL (right panels). Scale bar: 100 and 500 nm, respectively.

FIG. 16: CTB-ACE2 chewing gum study design. Phase I/II placebo-controlled, double-blind randomized ACE2 or placebo gum study for four days with 13 gums total, four gums each in days 1-3. An unstimulated whole saliva sample will be collected before eating or drinking or brushing teeth. Subjects will chew the CTB-ACE2 chewing gum/placebo gum (study product containing 2 grams of CTB-ACE2 or placebo) for 10 minutes and then immediately provide a post-treatment 2-5 mL saliva sample in pre-labeled salivary collection tubes. Viral load will be quantified by qPCR or protein (N or spike) quantitation.

### DETAILED DESCRIPTION OF THE INVENTION

As noted above, a high SARS-CoV-2 viral load is often detected in saliva (Li et al. Mol Oral Microbiol. 2020 35(4):141-145). In fact, salivary viral burden correlates with the severity of COVID-19 symptoms including the loss of taste and smell, and the virus replicates in salivary glands and oral mucous membranes (Huang et al. Nat Med. 2021 May; 27(5):892-903). Thus, the oral mucous membranes and saliva appear to be a high-risk route for SARS-CoV-2 transmission and viral inactivation within the oral cavity could be an important strategy to reduce viral infectivity. Mouth rinses including hydrogen peroxide and iodopovidone possess activity against coronaviruses in vitro, but their overall magnitude and duration of control due to short period of contact needs further investigations (Vergara-Buenaventura and Castro-Ruiz Brit J Oral Maxillofac Surg. 58(8):924-927).

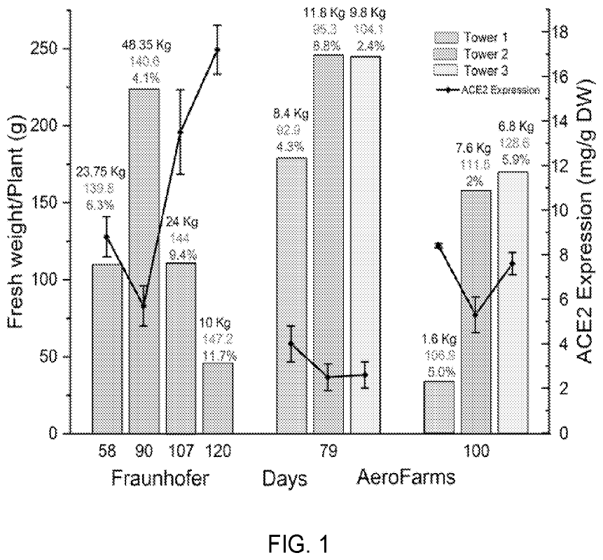

To test a novel debulking of target virus strategy in the oral cavity, the primary site of viral replication, SARS-CoV-2 virus trapping proteins CTB-ACE2 were expressed in chloroplasts and clinical grade plant material was developed to meet FDA requirements. Sec FIG. 1. Chewing gum (2 grams) containing plant cells expressed CTB-ACE2 up to 17.2 mg ACE2/g DW (11.7% leaf protein) have physical characteristics, taste/flavor like conventional gums and no protein is lost during gum compression. CTB-ACE2 gum efficiently (>95%) inhibited entry of Lentivirus-Spike or VSV-Spike pseudovirus into Vero/CHO cells, when quantified by luciferase or red fluorescence. Incubation of CTB-ACE2 microparticles reduced SARS-CoV-2 virus count in COVID-19 swab/saliva samples >95%, when evaluated by microbubbles (femtomolar concentration) or qPCR, demonstrating both virus trapping and blocking of cellular entry. COVID-19 saliva samples showed low or undetectable ACE2 activity when compared to healthy individuals (2582 vs 50126 ΔRFU; 27 vs 225 enzyme units), confirming greater susceptibility of infected patients for viral entry. CTB-ACE2 activity was completely inhibited by pre-incubation with SARS-CoV-2 RBD, offering an explanation for reduced saliva ACE2 activity among COVID-19 patients. Chewing gum with virus trapping proteins offers a general affordable strategy to protect patients from most oral virus reinfections through debulking or minimizing transmission to others.

The present inventor has surprisingly found that chewing gum comprising ACE2 is effective to minimize transmission and decrease infectivity by SARS-CoV-2 by inhibiting entry into human cells. Notably, these findings can also be extrapolated to other air borne viruses such as influenza, and measles. For treatment of flu for example, the chewing gum would comprise IAV blocking peptide which binds the flu virus and reduces concentrations of the same in the oral cavity and reducing influenza infectivity. Alternatively, the chewing gum can comprise FRIL obtained from lablab bean powder. FRIL binds both SARS-CoV2 and influenza viral surface proteins.

Although SARS-CoV-2 and flu are transmitted both nasally and orally, oral transmission is 3-5 orders of magnitude more likely than nasal transmission. The debulking of both viruses using viral trap proteins (CTB-ACE2, FRIL) expressed in plant cells is described herein. The viral trap proteins are delivered through chewing gum for optimal viral neutralization in the throat surface, the primary site of infection. In omicron nasopharyngeal (NP) samples, the microbubbling count (based on N-antigen) was significantly reduced by 20 μg of FRIL (p<0.0001) and 0.925 μg of CTB-ACE2 (p=0.0001). NP samples from 20 patients infected with delta or omicron strains, of these 17 samples had virus load below the detection level of spike protein in the RAPID assay. A dose-dependent 50% plaque reduction with 50-100 ng FRIL against Influenza strains H1N1, H3N2, and Coronavirus HCoV-OC43 was observed with both purified FRIL and lablab bean powder. In electron micrographs, large and densely packed clumps of overlapping influenza particles and FRIL protein were observed at 150 μg/mL FRIL concentrations but not in untreated virus particles in cell culture or particles purified via sucrose gradient. Collectively, these results provide proof of principle for use of chewing gum to deliver proteins to debulk oral viruses in the oral cavity and throat, thereby decreasing infection/transmission. Indeed, human papilloma virus, herpes simplex virus type I, Epstein-Barr virus and Kaposi's sarcoma associated herpes viruses are also orally transmitted, and thus carriers comprising molecules which bind surface proteins on these viruses could also be used to advantage to debulk the oral cavity and thus reduce viral transmission.

CTB is a transmucosal carrier and facilitates oral delivery of therapeutic proteins by forming pentameric structure and binding to gut GM1 epithelial receptors (Daniell et al 2019 Biotechnol. Adv. 37, 107413; Biomaterials. 2020 233:119750; Plant Biotechnol. 2021 J. 19: 430-447; Kwon and Daniell, Mol Ther. 2016 24(8): 1342-1350). CTB-ACE2 exhibits efficient binding to both GM1 and ACE2 receptors and thus effectively blocks the binding of the viral spike protein, particularly in oral epithelial cells that are enriched with both receptors (Xu et al. Int J Oral Sci. 2020 24; 12(1): 1-5). Direct binding of ACE2 to the SARS-CoV-2 spike proteins traps the virus particles and decreases infectivity. In keeping with this discovery, the invention provides a composition comprising CTB-ACE2 in a carrier suitable for oral administration, (e.g., chewing gum, long-lasting lozenge, tablet etc.) for inhibiting SARS-CoV-2 entry and transmission of the virus from the oral cavity. Notably, the tongue is a larger reservoir of ACE2 than buccal and gingival tissues (Xu et al; 2020, supra). ACE2 chewing gum represents a highly targeted approach for scavenging SARS-CoV-2 spike containing particles, which when exogenously released via mastication will provide greater sustainability than mouth rinses for effectively debulking SARS-CoV-2 viral load from the oral cavity.

In another application, a reduction of oral SARS-CoV-2 is necessary for infected patients who require emergency procedures, particularly dental procedures. CTB-ACE2 chewing gum could be used to decrease viral load in such patients, thereby providing greater protection to health care givers. In other approaches, CTB-ACE2 chewing gum could be consumed prophylactically, in public places, to augment or replace mask wearing in circumstances where mask use is not feasible or difficult, such as during eating. This strategy can also be employed to debulk viruses other than SARS-CoV-2 from the oral cavity as discussed above.

### Definitions

The sequences for expression of both native and codon optimized CTB-ACE2 are found in U.S. Pat. No. 10,806,775, which is incorporated herein by reference. See FIGS. 21A and 21B.

As used herein, the terms “administering” or “administration” of a composition to a subject includes any route of introducing or delivering to a subject a compound to perform its intended function. For treatment of air borne infections, the preferred route for administration can be oral, or intranasal. For other treatments that would benefit from CTB-ACE2 administration, other routes including parenteral (intravenously, intramuscularly, intraperitoneally, or subcutaneously), rectally, or topically, can be employed. Administering or administration includes self-administration and the administration by another.

As used herein, the terms “disease.” “disorder,” or “complication” refers to any deviation from a normal state in a subject.

As used herein, “infection” refers to the introduction and/or presence of a disease-causing, or pathogenic, organism into and/or in another organism, tissue or cell.

“Antimicrobial peptides (AMPs)” as used herein refers to host defense peptides (HDPs) which comprise a part of the innate immune response found among all classes of life. These peptides are potent, broad spectrum antibiotics which demonstrate potential as novel therapeutic agents. Antimicrobial peptides have been demonstrated to kill Gram negative and Gram positive bacteria, enveloped viruses, fungi and even transformed or cancerous cells. Unlike the majority of conventional antibiotics it appears that antimicrobial peptides frequently destabilize biological membranes, can form transmembrane channels, and may also have the ability to enhance immunity by functioning as immunomodulators. Suitable AMPs for use in the methods disclosed herein include, without limitation, protegrin, retrocyclin defensins, PGLA (frog skin), cecropins, apidaecins, melittin, MSI-99, bombinin, magainin, boceprevir, and telaprevir.

As used herein “Flt3 Receptor Interacting Lectin” or “FRIL” refers to a legume lectin that has a 48% sequence identity to the well-known concanavalin A (ConA), with a similar b-prism type-II fold and one carbohydrate-binding domain (CBD) per monomer. Previous studies have suggested that FRIL is a glucose/mannose-specific lectin based on its affinity for the monosaccharides mannose, glucose, and N-acetylglucosamine, with a strong preference for the a-anomeric configuration.

“Lipase” as used herein refers to a family of enzymes that catalyze the hydrolysis of fats. Lipase can be used to advantage to trap certain fungal species in a suitable carrier.

As used herein, “respiratory tract” means a system of cells and organs functioning in respiration, in particular the organs, tissues and cells of the respiratory tract include, lungs, nose, nasal passage, paranasal sinuses, nasopharynx, larynx, trachea, bronchi, bronchioles, respiratory bronchioles, alveolar ducts, alveolar sacs, alveoli, pneumocytes (type 1 and type 2), ciliated mucosal epithelium, mucosal epithelium, squamous epithelial cells, mast cells, goblet cells, and intraepithelial dendritic cells.

Orally-consumable products according to the invention are any preparations or compositions suitable for consumption, for nutrition, or for oral hygiene, and are products intended to be introduced into the human oral cavity, to remain there for a certain period of time and then to either be swallowed (e.g., food ready for consumption) or to be removed from the oral cavity again (e.g. chewing gums or products of oral hygiene). These products include all substances or products intended to be ingested by humans or animals in a processed, semi-processed or unprocessed state. This also includes substances that are added to orally-consumable products (e.g., active ingredients such as extracts, nutrients, supplements, or pharmaceutical products) during their production, treatment or processing and intended to be introduced into the human oral cavity.

As used herein, by the term “effective amount” “amount effective,” or the like, it is meant an amount effective at dosages and for periods of time necessary to achieve the desired result.

As used herein, the term “inhibiting” or “preventing” means causing the clinical symptoms of the disease state not to worsen or develop, e.g., inhibiting the onset of disease, in a subject that may be exposed to or predisposed to the disease state, but does not yet experience or display symptoms of the disease state.

As used herein, the term “expression” in the context of a gene or polynucleotide involves the transcription of the gene or polynucleotide into RNA. The term can also, but not necessarily, involves the subsequent translation of the RNA into polypeptide chains and their assembly into proteins.

A plant remnant may include one or more molecules (such as, but not limited to, proteins and fragments thereof, minerals, nucleotides and fragments thereof, plant structural components, etc.) derived from the plant in which the protein of interest was expressed. Accordingly, a composition pertaining to whole plant material (e.g., whole or portions of plant leafs, stems, fruit, etc.) or crude plant extract would certainly contain a high concentration of plant remnants, as well as a composition comprising purified protein of interest that has one or more detectable plant remnants. In a specific embodiment, the plant remnant is rubisco.

In another embodiment, the invention pertains to an administrable composition for treating or inhibiting transmission and/or infection by air borne viruses. In certain embodiments, the composition comprises a therapeutically effective amount of ACE2, CTB-ACE2 for treatment of Covid-19, or a combination thereof having been expressed by a plant and a plant remnant. The compositions of the invention may also be used to advantage prophylactically to reduce incidence of aerosolized transmission.

Importantly, this discovery can be used to advantage to inhibit the transmission and infectivity of other airborne viruses such as influenza. In this embodiment, the subject would consume chewing gum comprising influenza A virus (IAV) blocking peptides to debulk the oral cavity of influenza viral load.

Methods, vectors, and compositions for transforming plants and plant cells are taught for example in WO 01/72959; WO 03/057834; and WO 04/005467. WO 01/64023 discusses use of marker free gene constructs, each being incorporated herein by reference.

In a specific embodiment, plant material (e.g. lettuce material) comprising chloroplasts capable of expressing ACE2, CTB-ACE2, or HA, is homogenized, lyophilized and encapsulated. In one specific embodiment, an extract of the lettuce material is encapsulated. In an alternative embodiment, the lettuce material is powderized before encapsulation. Other useful plants include edible plants including without limitation, tomato, carrot and apple.

In some aspects, a pharmaceutical composition for debulking viral load in the oral cavity may be formulated as a chewing gum. The formulation of gum bases can vary substantially depending on the particular product to be prepared and on the desired masticatory and other sensory characteristics of the final product. By way of example, typical ranges of the gum base components include 5-80 wt. % elastomeric compounds, 5-80 wt. % natural and/or synthetic resins (elastomer plasticizers), 0-40 wt. % waxes, 5-35 wt. % softener other than waxes, 0-50 wt. % filler, and 0-5 wt. % of other ingredients such as antioxidants, colorants, and the like. The gum base may comprise about 5-95 wt. % of the total weight of the chewing gum, often from about 10-60 wt. % or from about 40-50 wt. %.

Often a buffer is used. Examples of buffers that may be used include tris buffers, amino acid buffers, carbonate, including monocarbonate, bicarbonate or sesquicarbonate, glycerinate, phosphate, glycerophosphate, acetate, glyconate or citrate of an alkali metal, such as potassium and sodium, e.g. trisodium and tripotassium citrate, or ammonium, and mixtures thereof. Other examples of buffers include acetic acid, adipic acid, citric acid, fumaric acid, glucono-Δ-lactone, gluconic acid, lactic acid, malic acid, maleic acid, tartaric acid, succinic acid, propionic acid, ascorbic acid, phosphoric acid, sodium orthophosphate, potassium orthophosphate, calcium orthophosphate, sodium diphosphate, potassium diphosphate, calcium diphosphate, pentasodium triphosphate, pentapotassium triphosphate, sodium polyphosphate, potassium polyphosphate, carbonic acid, sodium carbonate, sodium bicarbonate, potassium carbonate, calcium carbonate, magnesium carbonate, magnesium oxide, or any combination thereof.

The buffer may to some extent be microencapsulated or otherwise coated as granules with polymers and/or lipids being less soluble in saliva than is the one or more buffering agents. Such microencapsulation controls the dissolution rate whereby is extended the time frame of the buffering effect. The amount of buffer may range from 0 to about 15% and often ranges from about 0.5 to about 10% based on the total weight of the chewing gum.

Elastomers may be used to provide a rubbery, cohesive nature to the gum. Elastomers suitable for use in the gum base and gum may include natural or synthetic types. Elastomer plasticizers may be used to vary the firmness of the gum base. Their specificity on elastomer inter-molecular chain interaction (plasticizing) along with their varying softening points cause varying degrees of finished gum firmness and compatibility when used in base. This may provide more elastomeric chain exposure to the alkane chains of the waxes.

The elastomers employed in the gum base may vary depending upon various factors such as the type of gum base desired, the texture of gum formulation desired and the other components used in the formulation to make the final chewing gum product. The elastomer may be any water-insoluble polymer known in the art, and includes those gum polymers utilized for chewing gums and bubble gums. For example, polymers suitable for use in gum bases include, without limitation, natural substances (of vegetable origin) such as chicle gum, natural rubber, crown gum, nispero, rosidinha, jelutong, perillo, niger gutta, tunu, balata, guttapercha, lechi capsi, sorva, gutta kay, and the like, and mixtures thereof. Examples of synthetic elastomers include, without limitation, styrene-butadiene copolymers (SBR), polyisobutylene, isobutylene-isoprene copolymers, polyethylene, polyvinyl acetate and the like, and mixtures thereof.

Natural resins may be used according to the invention and may be natural rosin esters, often referred to as ester gums including as examples glycerol esters of partially hydrogenated rosins, glycerol esters of polymerized rosins, glycerol esters of partially dimerized rosins, glycerol esters of tally oil rosins, pentaerythritol esters of partially hydrogenated rosins, methyl esters of rosins, partially hydrogenated methyl esters of rosins, pentaerythritol esters of rosins, synthetic resins such as terpene resins derived from alpha-pinene, beta-pinene, and/or d-limonene, and natural terpene resins.

Resins may be selected from terpene resins, such as those derived from alpha-pinene, beta-pinene, and/or d-limonene, natural terpene resins, glycerol esters of gum rosins, tall oil rosins, wood rosins or other derivatives thereof such as glycerol esters of partially hydrogenated rosins, glycerol esters of polymerized rosins, glycerol esters of partially dimerized rosins, pentaerythritol esters of partially hydrogenated rosins, methyl esters of rosins, partially hydrogenated methyl esters of rosins or pentaerythritol esters of rosins and combinations thereof.

Other chewing gum ingredients may be selected from bulk sweeteners, flavors, dry-binders, tableting aids, anti-caking agents, emulsifiers, antioxidants, enhancers, absorption enhancers, buffers, high intensity sweeteners, softeners, colors, and combinations thereof. Non-limiting examples of emulsifiers include cyclodextrins, polyoxyethylene castor oil derivatives, polyoxyethylene alkyl ethers, macrogol alkyl ethers, block copolymers of ethylene and propylene oxides, polyoxyethylene alkyl ethers, polyoxyethylene glycols, polyoxyethylene sorbitan fatty acid esters, polyoxyethylene (20) sorbitan monostearates, polyoxyethylene (20) sorbitan monooleates, polyoxyethylene stearates, sobitan esters, diacetyl tartaric ester of monoglycerides, lactylated monoglycerides, and combinations thereof. The amount of emulsifiers often ranges from about 0.1% to about 25 wt. % based on the total weight of the chewing gum.

Petroleum waxes aid in the curing of the finished gum made from the gum base as well as improve shelf life and texture. Wax crystal size influences the release of flavor. Those waxes high in iso-alkanes have a smaller crystal size than those waxes high in normal-alkanes, especially those with normal-alkanes of carbon numbers less than 30. The smaller crystal size allows slower release of flavor since there is more hindrance of the flavor's escape from this wax versus a wax having larger crystal sizes. The compatibility of gum bases made using normal-alkanoic waxes is less when compared to gum bases made with iso-alkanoic waxes.

Petroleum wax (refined paraffin and microcrystalline wax) and paraffin wax are composed of mainly straight-chained normal-alkanes and branched iso-alkanes. The ratio of normal-alkanes to iso-alkanes varies.

The normal-alkanoic waxes typically have carbon chain lengths >C-18 but the lengths are not predominantly longer than C-30. The branched and ring structures are located near the end of the chain for those waxes that are predominantly normal-alkanoic. The viscosity of normal-alkanoic waxes is <10 mm²/s (at 100° C.) and the combined number average molecular weight is <600 g/mole.

The iso-alkanoic waxes typically have carbon lengths that are predominantly greater than C-30. The branched chains and ring structures are located randomly along the carbon chain in those waxes that are predominantly iso-alkanoic. The viscosity of iso-alkanoic waxes is greater than 10 mm²/s (at 100° C.) and the combined number average molecular weight is >600 g/mole. Synthetic waxes are produced by means that are atypical for petroleum wax production and are thus not considered petroleum wax. The synthetic waxes may include waxes containing branched alkanes and copolymerized with monomers such as, but not limited to propylene, polyethylene, and Fischer Tropsch type waxes. Polyethylene wax is a synthetic wax containing alkane units of varying lengths having attached thereto ethylene monomers.

Waxes and fats are conventionally used for the adjustment of the texture and for softening of the chewing gum base when preparing chewing gum bases. Any conventionally used and suitable type of natural and synthetic wax and fat may be used, such as for instance rice bran wax, polyethylene wax, petroleum wax (refined paraffin and microcrystalline wax), sorbitan monostearate, tallow, propylene glycol, paraffin, beeswax, carnauba wax, candelilla wax, cocoa butter, degreased cocoa powder and any suitable oil or fat, such as completely or partially hydrogenated vegetable oils or completely or partially hydrogenated animal fats.

Antioxidants prolong shelf life and storage of gum base, finished gum or their respective components including fats and flavor oils. Antioxidants suitable for use in gum base include butylated hydroxyanisole (BHA), butylated hydroxytoluene (BHT), betacarotenes, tocopherols, acidulants such as Vitamin C, propyl gallate, other synthetic and natural types or mixtures thereof.

A chewing gum may include other conventional components such as sweeteners, including bulk sweeteners, sugar sweeteners, sugar-substitute sweeteners, artificial sweeteners, high-intensity sweeteners, or a combination thereof. Bulk sweeteners may constitute from about 5 to about 95% by weight of the chewing gum, more typically about 20 to about 80% by weight, about 30 to 70%, or about 30 to 60% by weight of the gum.

Useful sugar sweeteners are saccharide-containing components commonly known in the chewing gum art including, but not limited to, sucrose, dextrose, maltose, dextrins, trehalose, D-tagatose, dried invert sugar, fructose, levulose, galactose, corn syrup solids, and the like, alone or in combination.

Sorbitol can be used as a non-sugar sweetener. Other useful non-sugar sweeteners include, but are not limited to, other sugar alcohols such as mannitol, xylitol, hydrogenated starch hydrolysates, maltitol, isomalt, erythritol, lactitol and the like, alone or in combination.

High intensity artificial sweetening agents can also be used alone or in combination with the above sweeteners. Non-limiting examples of high intensity sweeteners include sucralose, aspartame, salts of acesulfame, alitame, saccharin and its salts, cyclamic acid and its salts, glycyrrhizin, dihydrochalcones, thaumatin, monellin, sterioside and the like, alone or in combination. In order to provide longer lasting sweetness and flavor perception, it may be desirable to encapsulate or otherwise control the release of at least a portion of the artificial sweeteners. Techniques such as wet granulation, wax granulation, spray drying, spray chilling, fluid bed coating, conservation, encapsulation in yeast cells and fiber extrusion may be used to achieve desired release characteristics. Encapsulation of sweetening agents can also be provided using another chewing gum component such as a resinous compound.

Usage level of the artificial sweetener will vary considerably and will depend on factors such as potency of the sweetener, rate of release, desired sweetness of the product, level and type of flavor used and cost considerations. The active level of artificial sweetener may vary from about 0.001 to about 8% by weight, and often ranges from about 0.02 to about 8% by weight. When carriers used for encapsulation are included, the usage level of the encapsulated sweetener will be proportionately higher. Combinations of sugar and/or non-sugar sweeteners may be used if desired.

A chewing gum and/or gum base may include one or more fillers/texturizers, such as magnesium and calcium carbonate, sodium sulfate, ground limestone, silicate compounds such as magnesium and aluminum silicate, kaolin and clay, aluminum oxide, silicium oxide, talc, titanium oxide, mono-, di- and tri-calcium phosphates, cellulose polymers, such as wood, and combinations thereof.

A number of other well-known chewing gum components may be present, including but not limited to waxes, fats, softeners, fillers, flavors, anti-oxidants, emulsifiers, coloring agents, binding agents and acidulates. The chewing gum may be provided with an outer coating, such as a hard coating, soft coating, edible film-coating, or any combination thereof.

In some aspects, the trapping molecule which binds viral surface proteins, (e.g., CTB-ACE2, FRIL, IVA blocking peptide) is compounded along with other components of the gum base such that the molecule is substantially uniformly contained in the gum base. The molecules may be provided in a number of different forms, e.g. as a plant powder, lyophilized leaves, or on an adsorbent such as finely divided silicic acid, amorphous silica, magnesium silicate, calcium silicate, kaolin, clays, crystalline aluminosilicates, macaloid bentonite, activated carbon, alumina, hydroxylapatite, microcrystalline cellulose, or any combination thereof. In certain approaches, the molecules may be encapsulated to provide a desired controlled or sustained release thereof. An example of a chewing gum that provides for sustained release of nicotine is described in U.S. 2007/0014887, the disclosure of which is hereby incorporated by reference. This chewing gum can be adapted for the purposes disclosed herein.

A similar release profile may be achieved via an oral dosage form such as a tablet, capsule, or the like. For example, a tablet may have a core layer containing nicotine to provide a sustained release thereof, and an outer layer containing CBD to provide an immediate release thereof. Other combinations are possible. For example, one or both of the layers may contain both CBD and nicotine so that the respective active component(s) is released in both an immediate- and a sustained release manner.

The materials and methods below are provided to facilitate the practice of the methods disclosed herein.

### Materials and Methods for Example I

### Saliva Sample Collection

After informed consent under protocol #823392 approved by the University of Pennsylvania IRB, saliva and swabs of the nasopharynx and oropharynx were obtained from hospitalized patients with confirmed SARS-CoV-2 infection. Saliva was produced by patients spontaneously, and oropharyngeal and nasopharyngeal samples were collected with flocked nylon swabs (Copan Diagnostics) and eluted together in 2 ml of viral transport media. Saliva from healthy volunteers (confirmed SARS-CoV-2 negative) was collected following informed consent under protocol #842613 approved by the UPenn IRB. Specimens were stored at −80° C. until use.

### Plant Growth at Fraunhofer, Aerofarms, Biomass Harvest, Lyophilization

The hydroponic growth system at Fraunhofer USA includes multilevel growth racks illuminated to grow the transplastomic lettuce expressing CTB-ACE2. Growth trays contained rockwool, a lightweight hydroponic substrate that is manufactured by spinning molten basaltic rock into fibers, as the supporting substrate (Grodan, The Netherlands), Rockwool is designed to hold approximately 80% nutrient solution, with approximately 15% air space and 5% fibers, drains freely and encourages rapid uptake of nutrient solution by plant root systems. The nutrient solution comprised Peters Professional general purpose fertilizer at 200 parts per million with a 20-10-20 ratio of nitrogen, phosphate and potash (potassium oxide) (ICL Fertilizers, OH), and was supplemented with YaraLiva Calcinit (nitric acid, ammonium calcium salt) (Yara North America) at 102 parts per million. This nutrient concentrate was supplied via a Dosatron at a 1:100 injection ratio with potable water. Seedlings were spaced at 81/2 inches on a diagonal grid on the rockwool surface. Plants were exposed to LED lighting (Fluence SPYDRx) at an average of 368 μmols/m²·S on a 16-h day/8 hour night photoperiod under the conditions of temperature (24±2° C. day/20±2° C. night) and humidity (60±10%) in a growth room. Air flow across the plants was approximately 1.2 m/s.

At Fraunhofer USA, the transplastomic lettuce was repeatedly harvested from plants on days 58, 90, 107 and 120 post-seeding, typically at approximately 9 (8-11) hours into the day portion of the photoperiod. The leaves of lettuce plants were cut at the base of the petiole with operators wearing laboratory gloves, leaving the upper leaves intact on each plant and the fresh weight of biomass harvested was recorded. Leaves were rinsed in USP purified water, washed for approximately 5 minutes in a 200 parts per million chorine solution prepared using food grade calcium hypochlorite and then rinsed three consecutive times in USP purified water, with final chorine levels measured using chlorine sanitizer test strips to ensure levels are less than 4 parts per million. Excess water was then removed from leaf surfaces using a food grade centrifugal lettuce drier before being transferred to polyvinyl zip top bags. Bags were frozen on dry ice and then progressed to storage at ≤−60° C.-−80° C. Frozen lettuce leaves expressing CTB-ACE2 fusion proteins were freeze-dried in a lyophilizer (Ultra50, SP Scientific, Stone Ridge, NY). The lyophilizer is thoroughly cleaned and disinfected after each use and records collected on cycle parameters. If not proceeding directly to milling, the materials are placed into a storage bag containing a MiniPax® silica gel absorbent packet. The freeze-dried materials are stored in a dark steel cabinets, at room temperature storage room. The lyophilized leaves are ground in a grinder at single speed once for three seconds (optimized for minimal disrupted cells). The milled powder is poured over a 25 mesh (0.71 mm) hand sieve into the final sterile container closure. Any material that does not pass through the sieve is discarded. The materials are stored in sterile containers (FDA approved) inside steel cabinet in a dark, room temperature storage

The transplastomic ACE2 lettuce plants grown at Fraunhofer were harvested on 58, 90, 107, and 120 days after sowing seeds while, the AeroFarms grown plants were harvested on 79 and 100 days. Leaves were cut at the base of the petiole using scissors wearing sterile gloves, leaving the 3 to 5 upper leaves intact on each plant. Leaves are transferred to a polyvinyl zip top bag. Bags are labeled with the fresh weight, harvest date and plant line and frozen immediately. Biomass stored in the polyvinyl zip top bags are stored at ≤−60° C.-−80° C. Frozen lettuce leaves expressing CTB-ACE2 fusion proteins are freeze-dried in a lyophilizer (Ultra50, SP Scientific, Stone Ridge, NY). Shelves were pre-cooled to −40° C. and each of the fifteen shelves shelf loaded with ˜0.5 Kg of frozen leaves and lyophilizer probes were placed in the middle of plant materials in every other tray to monitor product temperature. Each lyophilization cycle starts with holding plant materials at −40° C. for 3 h. This was followed by drying cycle that consists of three stages: the first step of drying at −40° C. for ten min while at the second step temperature rises up to 25° C. for 3260 min. When the PVG/CM differential reaches to 43 mTorr, the Ultra 50 transits to the third step of drying where temperature drops to 22° C. and holds for eight hours. After the cycle ends, plant materials were unloaded and properly packed and weighed.

### Moisture Content

Moisture content was determined by Karl Fischer titration by which available water reacts with iodine and sulfur dioxide to form sulfur trioxide and hydrogen iodide. In brief, samples were weighed, and sample vials capped and placed in a Metrohm 850 KF Thermoprep and heated to 150° C. Vaporized water from samples was pumped into a Metrohm 851 Titrando at a rate of 50 mL per minute. Percent moisture was calculated from sample weight and reaction output, with Hydranal water standard (Honeywell) as a reference control. A recent model of freeze dryer (Virtis Ultra 50) has new gadgets to evaluate and monitor moisture content, which was not available in previous versions.

### Bioburden

Following harvest of biomass, leaves were washed in a 200 parts per million chlorine solution with triple rinse in USP purified water. Excess water was then removed from leaves and the tissue frozen and stored. The tissue was then freeze-dried in a lyophilizer (Ultra50, SP Scientific, Stone Ridge, NY), assessed for moisture content, ground, and sieved and again assessed for moisture content and for bioburden. Bioburden was evaluated according to USP <61> (microbiological examination of nonsterile products: microbial enumeration tests) and USP <62> (microbiological examination of nonsterile products: tests for specified microorganisms). Samples were assessed for aerobic microbial and fungal loads by plating serial dilutions in duplicate on each of trypticase soy agar and Sabouraud dextrose agar, respectively, and incubating for 3-5 days at 30-35° C. or 5-7 days at 20-25° C., respectively. For oral delivery, acceptance criteria were set according to USP <1111> (microbial examination of nonsterile products: acceptance criteria for pharmaceutical preparations and substances for pharmaceutical use) at ≤2×10³ cfu/g for total aerobic microbial count and ≤2×10² cfu/g for total yeasts and molds count.

### Total Protein Extraction and Quantification of ACE2

Expression levels of CTB-ACE2 was quantified by western blots using CTB antibody and standards as described previously (Daniell et al 2020), with suitable modifications. Five mg of lyophilized plant powder was extracted with 500 μl of protein extraction buffer (PEB) containing 100 mM NaCl, 10 mM EDTA (pH 8), 200 mM Tris (pH 8), 100 mM DTT, 1×PIC, 2 mM PMSF, and 10 mM β-mercaptoethanol. Samples were incubated at 4° C. vortex for 5 min and centrifuged at 14,000 rpm for ten min at 4° C. First supernatant was discarded, and pellet was re-suspended in 500 μl of PEB and incubated at 4° C. while vortexing for one hour. This was followed by sonication of samples at 80% amplitude for ten seconds on and 15 seconds off (3×) using sonicator 3000 (Misonix, Farmingdale, NY). Thereafter, samples were centrifuged 14,000 rpm for ten min at 4° C. and second supernatant was discarded. The pellet was re-suspended in 200 μl of PEB buffer and sonicated as described above. The total protein concentration was quantified by Bradford assay (Bio-rad Laboratories, Hercules, CA) and homogenized proteins were heated at 70° C. in 1× Laemmli buffer for 15 min and run on 10% SDS-polyacrylamide page. For each sample, 25 and 50 ng of total protein was loaded on western blot gel and anti-CTB antibody (1:10,000) (Gen Way Biotech, San Diego, CA), goat anti rabbit IgG-HRP secondary antibody (1:4000) (Southern Biotechnology, Birmingham, AL), and the precision plus protein standard (Bio-rad Laboratories, Hercules, CA) were used for all western blot analyses. The ACE2 proteins were detected by Supersignal west Pico Chemiluminescent substrate (Thermo Fisher Scientific, Waltham, MA) on iBright Western Blot Imaging Systems (Thermo Fisher Scientific, Waltham, MA). The ACE2 protein quantification was carried out according CTB standard concentration using iBright Analysis Software (Thermo Fisher Scientific, Waltham, MA).

### ACE2 Enzyme Activity Assay in Plant Extract and Saliva

ACE2 activity assay was performed using saliva of SARS-CoV-19 infected and healthy people. Saliva samples were prepared at 1:5 dilution. 75 μl of this prepared sample was added to a black 96-well microtiter plate containing 25 μl of ACE2 buffer (1 mol/L NaCl, 75 mmol/L Tris HCl, pH 7.5, and 50 μmol/L ZnCl2), and 20 μmol/L of ACE2-specific fluorogenic peptide substrate VI Mca-APK (Dnp) (R&D Systems, Minneapolis, MN). The enzymatic activity was recorded for 90 minutes at 5 min intervals at 28° C. with optic position top and gain extended; with excitation at 340 nm and emission at 405 nm. After the reading the relative fluorescence unit at each time point was plotted. ΔRFU was calculated by subtracting data of timepoint 0 minutes from the data of time point 90 minutes. Total soluble protein in the remaining saliva sample was determined by Bradford assay using standard Daniell lab's protocol. The final ACE-2 enzyme activity was calculated as pmol/min/mg (mU/mg)=Δpmol/90 min/mg total protein.

### Microbubbling SARS-CoV-2 Antigen Assay

One hundred and fifty μL patient samples was added to each powder tube and vortexed. Tubes were then incubated at 4° C. for one hour while rotating. Following incubation, tubes were vortexed again, then spun at 14,000 RPM for 20 minutes at 4° C. Tubes were recovered and 120 μL of supernatant was first mixed with virus lysis buffer of 10% TWEEN 20 (100×, 1.2 μL) and 100× protease inhibitor cocktail (1.2 μL) and incubated at room temperature for 30 min. The lysed sample was then tested the Microbubbling SARS-CoV-2 Antigen Assay (Chen H. et al. 2019, 2021). Briefly, sample solutions (100 μL) were incubated with suspensions of 500,000 capture antibody functionalized magnetic beads, on a roller (12 rpm) at room temperature for 30 min. The beads were then separated using magnets and washed 3 times with PBS buffer pH 7.4 containing 0.01% TWEEN 20, and then resuspended in 100 μL of 250 ng/mL biotinylated detection antibody in PBS containing 1% BSA, on a roller (12 rpm) at room temperature for 30 min. The beads were then separated using magnets and washed 3 times with PBS buffer pH 7.4 containing 0.01% TWEEN 20, and then resuspended in 100 μL of 1 μg/mL NeutrAvidin functionalized PtNP in PBS containing 1% BSA, on a roller (12 rpm) at room temperature for 30 min. The beads were then separated using magnets and washed 3 times with PBS buffer pH 7.4 containing 0.01% TWEEN 20 and resuspended in 100 μL of 30% H₂O₂. The magnetic beads slurries were then added into the chambers of the microbubbling microchips. The microbubbling microchips were placed on a neodymium disc magnet for 1 min to pull down the beads to the bottom of the microchips. Microbubbles on the microbubbling microchips were imaged using an iPhone 11 or an iPad with the uHandy mobilephone microscope (9×, 5 mm focusing length, Aidmics Biotechnology Co. Taipei, Taiwan).

### Lentivirus-Spike Neutralization Assay

Lentiviral particles were prepared in 293 FT cells according to Crawford et al. (Crawford, Katharine H D, et al. “Protocol and reagents for pseudotyping lentiviral particles with SARS-CoV-2 spike protein for neutralization assays.” Viruses 12.5 (2020): 513). Vero cells were seeded overnight at 1.25×10⁴ cells/well in a 96-well plate in a humidified incubator at 37° C. and 5% CO₂. ACE2 gum or corresponding placebo was combined with 800 μL of growth medium containing enough viral particles to generate signals that were at least 200-fold over the background luciferase activity. After gentle mixing at 4° C. for 1 h, the supernatant was collected by 20-min centrifugation at 14,000 rpm at 4° C. and 100 μL of the supernatant was added to each well, followed by an additional 110 μL of fresh media containing 5 μg/mL polybrene. After 24-h treatment, cells were processed with the Bright-Glo Luciferase Assay System (Promega) according to the manufacturer's recommendation, and luminescence was measured on a microplate reader.

### VSV-S Pseudotypes Neutralization Assay

VSV-S pseudotype particles were generated using a vesicular stomatitis virus (VSV) platform described previously (1, 2). Briefly, pseudotyped VSV virions that incorporate the SARS-CoV-2 Spike protein into their envelopes were produced by transfection of HEK293T cells with pCG1 SARS-CoV-2 S delta18 expression plasmid encoding a codon optimized SARS-CoV-2 S gene with an 18-residue truncation in the cytoplasmic tail (kindly provided by Paul Bates and Stefan Pohlmann) (3). At 30 hours post transfection, the SARS-CoV-2 Spike expressing cells were transduced for 4 hours with VSVΔG-RFP pseudotypes. The viral inoculum was removed, and the cells were washed twice with PBS 1× to remove unbound virus. At 28 hours post transduction, the media containing the VSV-S pseudotypes was harvested and clarified by centrifugation twice at 4,000 rpm for 15 minutes. VSV-S pseudotypes were aliquoted and stored at −80° C. until use in ACE2 neutralization studies.

Vero E6 cells were seeded at 1×10\hat{ }4 cells per well in a 96 well plate and incubated overnight at 37° C. 100 ul of VSV-S pseudotype particles (˜1.2×10⁴ red fluorescent units) in media were mixed with 250 ng of CTB-ACE2 or 5 mg, 10 mg, 20 mg or 50 mg ACE2 gum powder and rotated end over end for 30 minutes at 4° C. Samples were centrifuged at 14.8K rpm for 20 minutes at 4° C. to pellet the gum powder, and the supernatant was added to Vero cells for 1 hour at 37° C., swirling every 15 minutes. Virus inoculum was removed, and the cells were washed twice with PBS 1× to remove unbound viral particles. At 24 hours post transduction, red fluorescent protein (RFP) expression was visualized and quantified on a fluorescent microscope. RFP expression in each condition was measured in two technical replicates performed in two independent experiments. Inhibition of VSV-S entry was calculated relative to the untreated VSV-S control and statistical significance was analyzed by student t-test.

### ddPCR Assay

Saliva samples of 15 COVID-19 patients were collected by the University of Pennsylvania medical staffs. To avoid the risk of viral infection and false positive results potentially due to the laboratory contamination, all the experiments were done inside a BSL2 enhanced biosafety cabinet. 140 μl of saliva were mixed and incubated with 100 mg ACE2 gum powder for 1 hr at room temperature while rotating, then spun at 14,000 rpm for 20 min. Total RNA was extracted from the supernatant using QIAamp viral RNA mini kit (Qiagen) following manufacturer's instruction. ddPCR was performed in duplicates via the COVID-19 digital PCR detection kit (Biorad). All the procedures follow the manufacture instructions of the QX200 Droplet Digital PCR System using supermix probe (Bio-Rad). The kit allows the detection of the regions of nucleocapsid N1, N2 gene, and Rnase P gene positive reference gene. Twenty-two microliters of each reaction mix was converted to droplets with the QX200 droplet generator (Bio-Rad). Droplet-partitioned samples were then transferred to a 96-well plate, sealed and cycled in a T100 Thermal Cycler (Bio-Rad) under the following cycling protocol: 95° C. for 10 min (DNA polymerase activation), followed by 40 cycles of 94° C. for 30 s (denaturation) and 60° C. for 1 min (annealing) followed by an infinite 4-degree hold. The cycled plate was then transferred and read in the FAM and HEX channels using the QX200 reader (Bio-Rad). Data was analyzed using QuantaSoft analysis Pro 1.0.596 software (Bio-Rad).

The following examples are provided to illustrate certain embodiments of the invention. They are not intended to limit the invention in any way.

### Example I

### Debulking Virus Using ACE2 or HA in Chewing Gum to Decrease Oral Virus Transmission and Infection Clinical Grade ACE2 Plant Production at Fraunhofer USA, Aerofarms

CTB-ACE2 plants were created as reported in a previous publication (Daniell et al 2020, supra). Seeds from the same batch were provided to Fraunhofer and Aerofarms and plants were grown as described in the methods section. While growth conditions were different, biomass yield per plant was similar on 80-90 days but declined as plants grew older. However, the most dramatic difference was in the expression level of CTB-ACE2; while the maximum expression for plants grown at Aerofarms was <2 mg CTB-ACE2/g dry weight, the expression level at Fraunhofer USA ranged from 15-22 mg/g dry weight, which likely represents the highest expression level achieved to date in engineered leaves (FIG. 1 and Table 1). However, irrespective of growth conditions, highest expression was observed in oldest plants when lowest biomass was harvested at both facilities. Therefore, delayed single final harvest could further improve protein drug production and yield. Total protein based on dry weight was also higher at Fraunhofer (140-147 mg/g DW) than plants grown at AeroFarms (93-129 mg/g DW), suggesting that the different nutrient solutions influence protein synthesis. Higher level expression reduces the amount of plant powder required for chewing gum or oral delivery of therapeutic ACE2 and therefore is an important production metric.

TABLE I
Fresh harvested biomass, moisture content, total protein content, and ACE2 content of CTB-ACE2 plants
grown at Fraunhofer and Aerofarms related to their age and growth condition. The values of CTB-ACE2
expression are mean ± SD (n = 3). The values for total leaf protein are the mean ± SD (n = 8).

| | | | | | | CTB-ACE2 | Total Protein | % CTB-ACE2 |
|---|---|---|---|---|---|---|---|---|
| | Growth | Age | Fresh Weight/ | Total fresh | Moisture | Expression | content (mg/g | content/Total |
| Entity | Condition | (Days) | Plant (g) | biomass (Kg) | Content % | (mg/g DW) ± SD | DW) ± SD | protein content |
| Fraunhofer | Standard | 58 | 110 | 23.8 | 5.6 | 8.8 ± 0.9 | 139.8 ± 4.2 | 6.3 |
| | | 90 | 224 | 48.4 | 10.9 | 5.7 ± 0.9 | 140.6 ± 6.6 | 4.1 |
| | | 107 | 111 | 24 | 7.4 | 13.5 ± 1.9 | 144.0 ± 7.6 | 9.4 |
| | | 120 | 46 | 10 | 6.98 | 17.2 ± 1.1 | 147.2 ± 6.0 | 11.7 |
| AeroFarms | Tower 1 | 79 | 179 | 8.4 | 5.6-6.3 | 4.0 ± 0.8 | 92.9 ± 1.5 | 4.3 |
| | | 100 | 34 | 1.6 | 6.2 | 8.4 ± 0.1 | 95.3 ± 2.3 | 8.8 |
| | Tower 2 | 79 | 245 | 11.8 | 7.4 | 2.5 ± 0.6 | 104.1 ± 6.7 | 2.4 |
| | | 100 | 158 | 7.6 | 5.3 | 5.3 ± 0.8 | 106.9 ± 5.6 | 5.0 |
| | Tower 3 | 79 | 245 | 9.8 | 6.5 | 2.6 ± 0.6 | 111.5 ± 6.8 | 2.3 |
| | | 100 | 170 | 6.8 | 5.6 | 7.6 ± 0.5 | 128.6 ± 5.0 | 5.9 |

### Chewing Gum Preparation

Chewing gum tablets containing ground plant powder were prepared by Per Os Biosciences (Hunt Valley, MD) by compression process but not the traditional gum manufacturing process which requires higher temperature and extrusion/rolling that introduces variability in the concentration of the active ingredient. Gum tablets contained the gum base (28.2%), maltitol (20.4%), sorbitol (13%), xylitol (13%), isomalt (13%), natural and artificial flavors, magnesium stearate (3%), silicon dioxide (0.43%), stevia (0.65%) and freeze-dried plant cells expressing ACE2 in order to offer the best flavor, taste, softness and compression. The gum tablet chews and performs exactly like the conventional chewing gum based on physical characteristics. Freeze-dried plant cells were ground with five and ten pulses to release more protein in the supernatant and retain less within intact plant cells that might require mechanical grinding by teeth while chewing. Two different concentrations of ACE2 gum powder were made in 2 gram chewing gum tablets and placebo gum that didn't include CTB-ACE2 freeze-dried plant cells.

### Neutralization Assay with SARS-CoV-2 Spike-Pseudotyped Lentiviral Particles

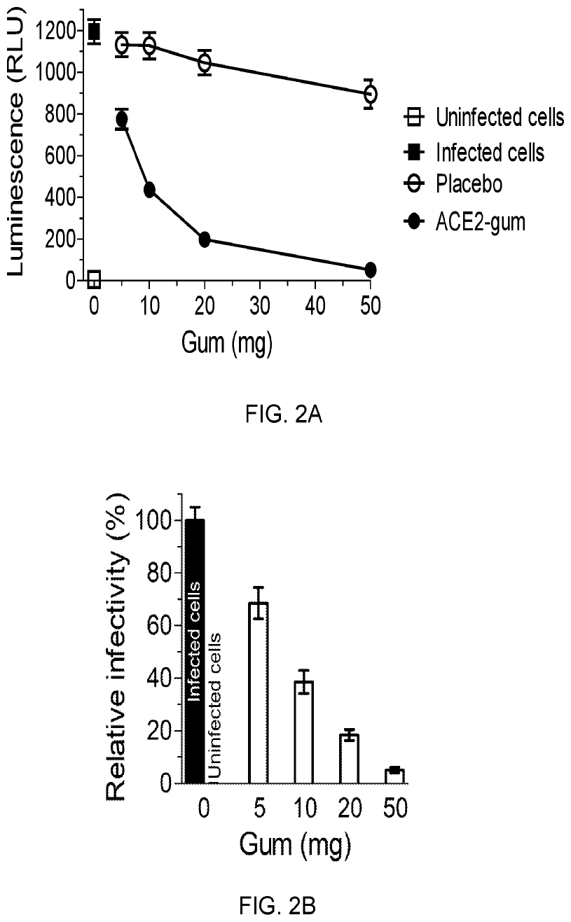

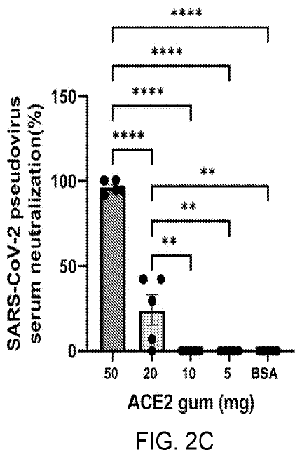

CTB-ACE2 has the potential to effectively bind to both the GM1 and ACE2 receptor binding sites located in close proximity on the human cell surface and thereby prevent viral entry into human cells. Therefore, we employed a SARS-CoV-2 pseudotyped lentivirus (also referred to as lentivirus particles) in order to determine the effectiveness of ACE2 gum in neutralizing spike-mediated viral infection. Lentiviral particles pseudotyped with the viral spike protein and harboring the pseudoviruses expressing a luciferase reporter gene were used to infect CHO cells expressing human ACE2. SARS-CoV-2 spike glycoprotein pseudotyped viruses expressing luciferase were incubated with ACE2 gum at the indicated concentration for 90 minutes at room temperature. Following centrifugation, virus-containing supernatant was incubated with ACE2-expressing CHO cells for 72 hrs, and viral infectivity was measured via luciferase As shown in FIG. 2A, the incubation of lentivirus with increasing amounts of ACE2 gum significantly reduced luciferase activities in a dose-dependent manner, indicating the effective neutralization of spike-mediated infection. Specifically, the comparison of the ACE2 gum treatments to their corresponding placebos showed ACE2 to significantly suppress spike-mediated infection. For example, 50 mg of ACE2 gum was effective at decreasing infectivity by approximately 95% compared to the corresponding placebo (FIG. 2B).

Data shown in FIG. 2C are representative of three independent experiments (N=2-3 samples). Bars represent the condition mean, symbols represent the average of duplicate or triplicate assays, error bars represent the SEM. The incubation of pseudotyped lentivirus with increasing amounts of CTB-ACE2 gum significantly reduced luciferase activities in a dose-dependent manner; 50 mg CTB-ACE2, highest inhibition (p<0.0001****) was observed (FIGS. 2A, 2B and 2C), confirming ability of this protein to block entry of lentivirus spike protein into CHO cells either by direct binding to the spike protein or by binding of CTB-ACE2 to ACE2/GM1 receptors on CHO cells.

### VSV-S Pseudotype Entry is Inhibited by Recombinant CTB-ACE2 and ACE2 Gum Powder

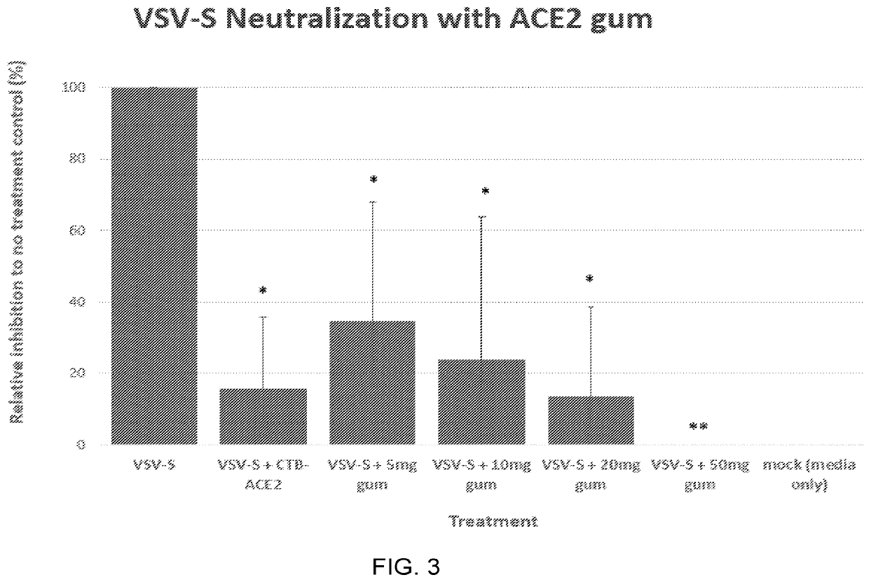

The RFP-expressing VSV-S pseudotype particles utilize the SARS-CoV-2 Spike (S) protein to bind and enter cells. We assessed whether our purified recombinant CTB-ACE2 protein or ACE2 gum powder would bind to VSV-S particles and inhibit viral particle entry into Vero cells. In repeated experiments, we found that VSV-S entry was inhibited with the addition of CTB-ACE2 by approximately 85% compared to untreated controls (FIG. 3), confirming the anti-viral effect of our recombinant protein on entry of the VSV-S pseudotypes. We also observed a dose dependent inhibition of entry when VSV-S particles were incubated with increasing concentrations of our ACE2 gum powder (FIG. 3). Indeed, representative images from fluorescent microscopy highlight clear and significant differences in the number of cells expressing RFP in control vs. 20 mg of ACE2 gum powder samples. Taken together, these data show that CTB-ACE2 protein and ACE2 gum powder traps the VSV-S pseudotype particles, thus decreasing viral entry either by binding directly to the spike protein or by binding to ACE2/GM1 receptors on the surface of Vero cells.

### Evaluation of SARS-Cov-2 in Swab Samples by Microbubbles and Gum

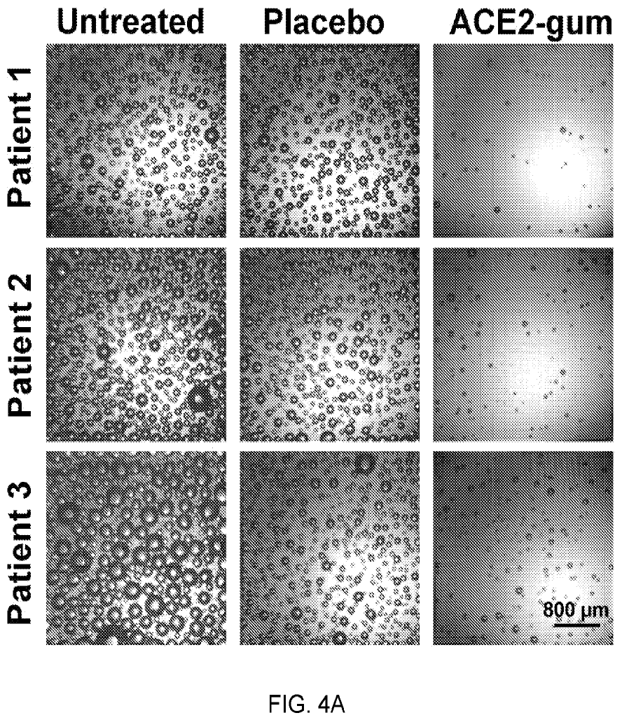

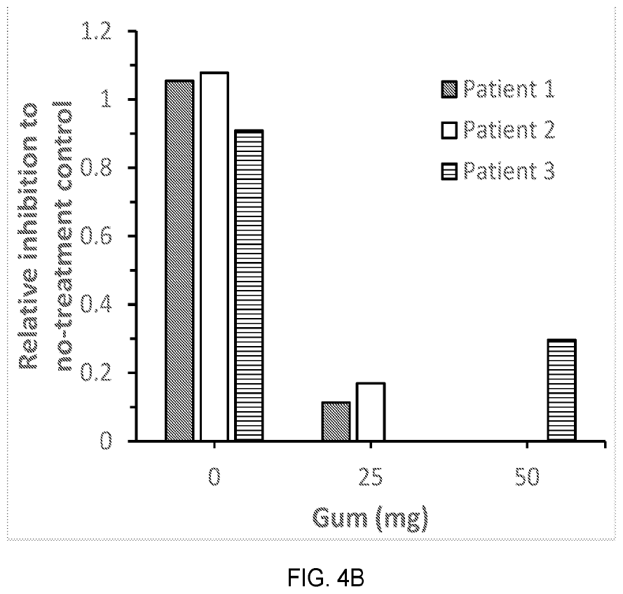

Several deidentified nasopharyngeal (NP) swab samples collected from patients positive for SARS-CoV-2 nucleic acid and nucleocapsid antigen were used to evaluate the neutralization of viral particles by ACE2-gum. The Microbubbling SARS-CoV-2 Antigen Assay detects the nucleocapsid antigen of SARS-CoV-2 at femtomolar concentration (see references added in the references section Chen et al. Ange Chemie 31, 14060-14066 (2019); Chen et al. MedRxiv doi: 10.1101/2021.03.17.21253847). The number and size of microbubbles correlates with the amount of nucleocapsid antigen in the sample. Samples of patient 1 and patient 2 were treated with 25 mg gum per 150 μL sample and the sample of patient 3 was treated with 50 mg gum per 150 μL sample. Samples were then tested using the Microbubbling SARS-CoV-2 Antigen Assay. As shown in FIG. 4, the incubation of swab samples with ACE-2 gum significantly reduced the microbubble counts compared with placebo gum and the untreated group, indicating ACE-2 gum decreased the amount of nucleocapsid antigens in the patients' samples. The percent of reduction ranged from 70% to 89%. In contrast the placebo gum when compared to the untreated group has less significant variation (p value 0.892). ACE2 gum decreased the amount of nucleocapsid antigens in COVID-19 patient samples even at very low concentration (25 mg), whereas each gum weights 2 grams. These results confirm direct binding of ACE2 to the SARS-CoV-2 spike proteins and entrapment in pentameric insoluble microparticles of CTB-ACE2 facilitate removal of bound viral particles by centrifugation.

### ACE2 Activity, in Control and COVID-19 Patient Saliva

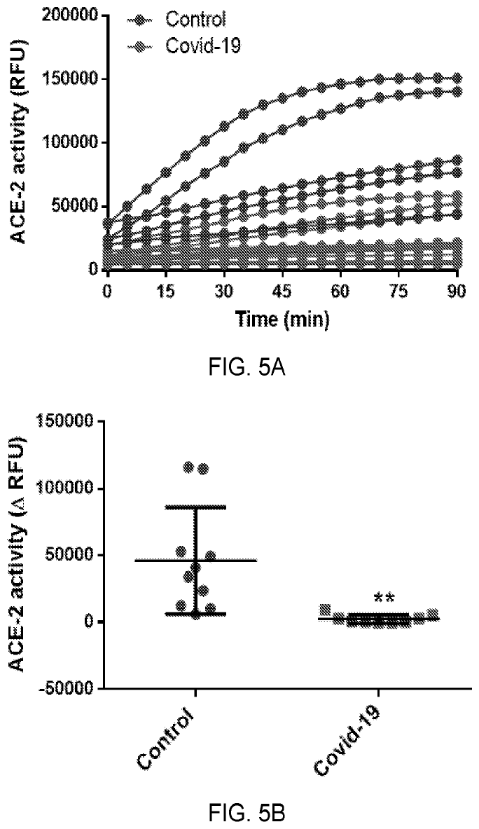

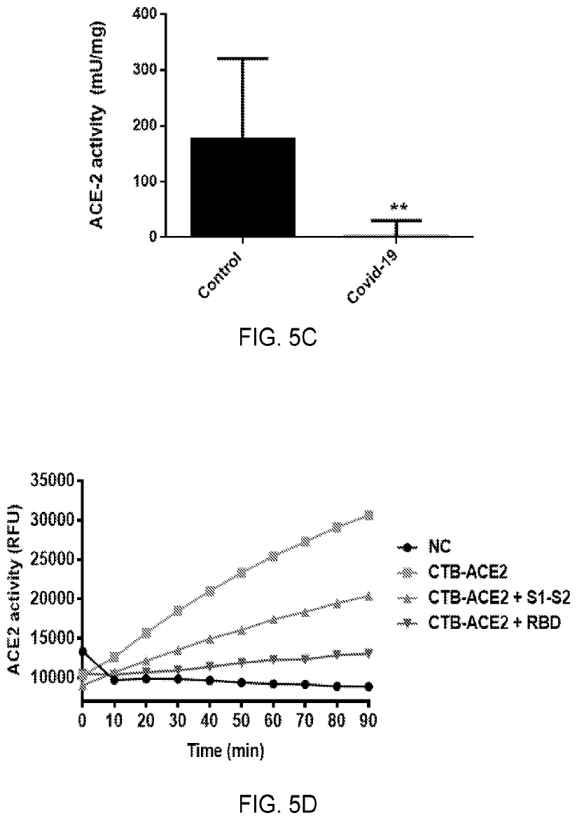

We obtained saliva samples from 10 COVID-19 patients (age range) and 10 healthy controls. ACE2 activity was markedly reduced in COVID-19 patients compared to controls (FIG. 5A-C). We observed a drastic reduction in ACE2 activity in viral laden saliva collected from 10 COVID-19 patients compared to healthy individuals (2582±439.82 vs 71356±2116 ΔRFU: 27.63±9.52 vs 257±7.89 mU/mg enzyme activity units) The fluorescent cleaved product of ACE 2 (Mca-YVADAPK) steadily increased up to ninety minutes in control saliva samples. While all Covid-19 saliva samples showed a similar low and almost undetectable ACE2 activity, one sample stood out as an outlier with enzyme activity (38504±9688 ΔRFU: 236.4±60.28 mU/mg enzyme activity units) similar to control saliva, which is excluded in FIG. 4B, C. This patient is asymptomatic based on the patient chart and didn't develop the COVID-19 disease, although PCR data showed presence of SARS-CoV-2. Therefore, saliva ACE2 activity is a powerful biomarker to distinguish symptomatic from asymptomatic COVID-19 patients. Slight variations among ACE2 activity among healthy patients based on demographic data is currently under investigation in patients of different age groups or of ethnic background.

### The Activity of Full Length CTB-ACE2 is Inhibited by SARS-CoV-2 Spike Protein

To investigate ACE2 mechanistic reasons for decrease in ACE2 activity in COVID-19 saliva, inhibition by SARS-CoV-2 spike proteins, in vitro enzymatic assay was performed using full length CTB-ACE2 in the presence or absence of the SARS-CoV-2 spike protein (RBD, S1-S2). CTB-ACE2 was extracted from CTB-ACE2 transplastomic lyophilized leaf powder and 20 μg of protein extracts was used for fluorogenic kinetic assay. The fluorescent cleaved product of CTB-ACE2 protein extracts (Mca-YVADAPK) gradually amplified for up to ninety minutes, demonstrating that the ACE2 was enzymatically active (FIG. 5D). However, this activity was partially blocked when CTB-ACE2 was pre-incubated with 10 μg of SARS-CoV-2 S1-S2 spike protein for 30 mins at RT (FIG. 5D). In fact. ACE2 activity showed the complete inhibition by the pre-incubation (30 min) of 10 μg SARS-CoV-2 RBD. Collectively, this finding suggests that SARS-CoV-2 spike proteins bind to the full-length ACE2 through the RBD^{[4,5,6]} and thus diminished the ACE2 activity.

### Evaluation by Debulking of SARS-CoV-2 in Saliva by ddPCR

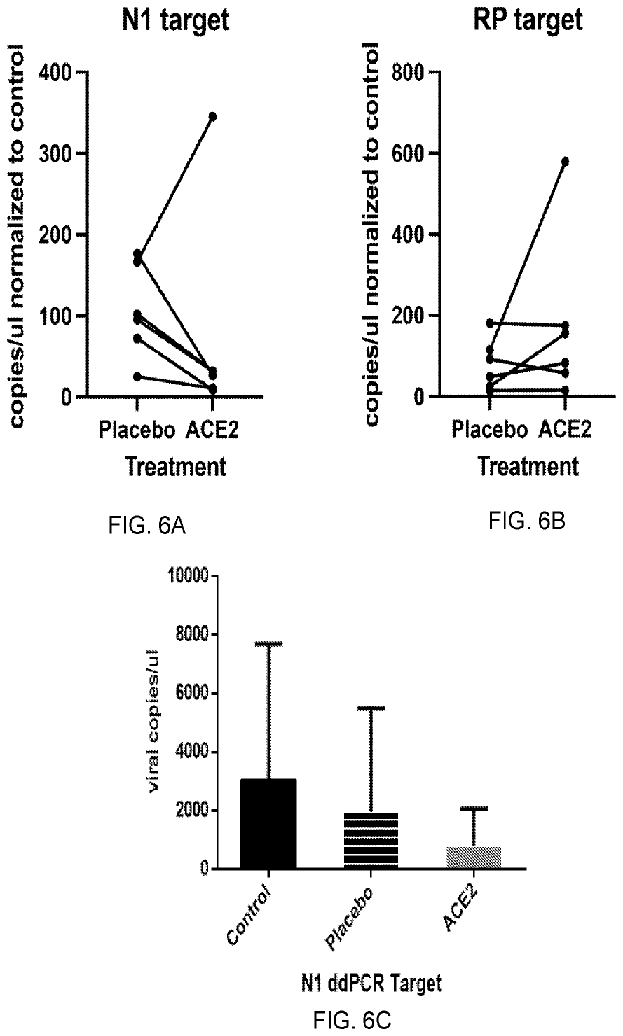

Several deidentified saliva samples collected from patients positive for SARS-CoV-2 were used to evaluate debulking of the viral particles by ACE2 or placebo gum using ddPCR. Unlike the microbubble assay where actual viral particles are measured, ddPCR amplified the viral RNA and therefore actual copies of viral RNA present in patients is not measured. While PCR amplification is used to increase sensitivity of saliva tests, it is not quantitative. Indeed. PCR amplification hasn't yet been used to predict the severity of COVID-19 disease. Considering these limitations with PCR approach, we evaluated this approach to measure debulking of viral in the saliva. Despite amplification, in most samples examined, there was 2-4 fold reduction with the placebo and >10-fold reduction with ACE2 gum, almost to the lowest number of copies that could be reliably measured by ddPCR. As shown in FIGS. 6A and 6B, the incubation of saliva samples with ACE-2 gum significantly reduced SARS-Cov2 copies when compared with placebo gum and the untreated group. The percent of reduction ranged >90% in most treated samples. See FIG. 6C.

### Discussion

In healthy human lungs, ACE2 is primarily expressed in type II alveolar epithelial cells that produce surfactants to protect alveoli from collapsing and possess tight junctions that limit fluid transudation. ACE2 is an integral part of the renin-angiotensisn system (RAS), and cleaves angiotensin II (Ang II), which causes vasoconstriction, inflammation, hypercoagulation and fibrosis (Gheblawi et al. Circ Res. 2020; 126:1456-1474), to produce the anti-inflammatory, cytoprotective angiotensin 1-7 (Ang 1-7) peptide. Human ACE2 exists in both the soluble (sACE2) and membrane associated ACE2 (mACE2) forms, the latter being the most predominant form (Rahman et al. Rev Med Virol. 2021; 1-12; Anand et al. Viruses 2020, 12, 1104; Batlle et al. 2020 Clinical Science 134: 543-545). Low abundance of sACE2, short life and host cell entry as SARS-CoV-2-sACE2 complex have been explained to be one among the reasons for comorbid COVID-19 patients who fail to gain protection (Rahman et al 2021, surpra). In case of therapeutic ACE2, additional infusion could supplement the lost sACE2 and help balance RAS by prevention of downregulation in COVID-19 patients (Zoufaly et al. Lancet Respir Med. 2020; 8:1154-1158 or in pulmonary hypertension disease model oral ACE2 results in an attenuation of pulmonary hypertension development with decreases in right ventricular (RV) hypertrophy, RV systolic pressure, total pulmonary resistance and pulmonary artery remodeling (Daniell H et al. 2020, supra; Shenoy et al. 2014 Hypertension 64, 1248-1259). In contrast to injected truncated (transmembrane deleted) sACE2 (Zoufaly et al. 2020, supra), full length oral CTB-ACE2 accumulates in the lungs at 10-fold higher concentrations than in the plasma upon oral delivery of bioencapsulated plant cells (Daniell et al. 2020 supra, 2021, supra), offering yet another approach to treat COVID-19 patients. Indeed, oral delivery of protein drugs bioencapsulated in plants reduces cost by elimination of prohibitively expensive fermentation, purification, cold chain for transportation/storage and sterile injections (Daniell et al 2019, supra; 2021, supra; Park et al. 2020 Biomaterials 233, 119591).

ACE2 chewing gum was investigated for the ability to trap SARS-CoV-2 in order to debulk the virus and reduce oral transmission. While entry of SARS-CoV-2 into human cells through the ACE2 receptor has been widely reported, the requirement for GM1 co-receptor has been poorly studied (Fantini et al. Int J Antimicrob Agents. 2020; 56: 106020) Likewise, published literature predominantly focuses on ACE2 receptor while the role of soluble ACE2 is poorly understood. Indeed, SARS-CoV-2 has greater binding affinity to monomeric soluble ACE2 than other known coronaviruses (Anand et al. 2020, supra).

The experiments in this study were designed for: 1) binding of CTB-ACE2 directly to the spike protein and trapping virus particles in the gum base or in the pellet after centrifugation; 2) CTB-ACE2 forms pentameric insoluble nanoparticles (and this should facilitate trapping of the virus particles; 3) SARS-CoV-2 requires both ACE2 receptor and GM1 co-receptor for cellular entry-CTB-ACE2 can saturate both these receptors due to high affinity of CTB to GM1 receptor, thereby facilitating virus neutralization studies in Vero cells using VSV or Lentivirus engineered to express spike proteins or prevent entry into human oral epithelial cells. Therefore, sACE2 could compete for ACE2 receptor binding site with SARS-CoV-2 and act as “decoy” and also directly bind to SARS-CoV-2 spike protein, preventing entry into human cells (Daniell et al 2021, supra; Batle et al. 2020, supra). Indeed, a recent study shows that the spike protein engineered in a pseudovirus damages vascular endothelial cells by downregulating ACE2 and consequently inhibiting mitochondrial function (Lei et al. Circulation Research 2021:128:1323-1326). Therefore, trapping SARS-CoV-2 and decreasing spike protein levels using the ACE2 chewing gum is of paramount therapeutic and prophylactic importance.

It should be noted that there is some placebo effect and the intensity of this effect depends on viral particle density tested in our study. In silico screening of 48 sugar alcohol compounds identified three-sugar alcohol compounds with maximum binding affinity with viral proteins (sorbitol, mannitol, and galactitol), especially high affinity of Ebola VP40 to sorbitol based on the binding energy and the number of hydrogen bond interactions (Nagarajan et al 2019 Molecular Biology Reports 46: 3315-3324). ACE2 chewing gum contains maltitol (20.4%) and 13% each of sorbitol and xylitol. Sugar-free chewing gum products made by Wrigley's. Mondelaz and Hershey's contain various combinations of xylitol, sorbitol, isomalt and maltitol. Two major chewing gum brands, Stride (Mondelaz) and Ice Breakers (Hershey's) include all four of these sugar alcohols. Therefore, those who chew gums during the pandemic may have some advantage in reducing viruses in their oral cavity but, notably addition of ACE2 to the gum base dramatically enhances virus particle trapping and neutralization

### Pseudovirus Evaluation

VSV particles expressing the S protein of SARS-CoV-2 can be used to accurately mimic entry of this coronavirus into ACE2-expressing cells. Notably, this assay can be performed rapidly and at the BSL-2 level to provide and initial evaluation of the efficacy of entry inhibitors. Here, we used this approach to show that recombinant CTB-ACE2 and increasing concentrations of ACE2 gum powder effectively inhibited entry of VSV-S particles compared to controls. Consistently, the ACE2 gum powder also showed effective inhibition of infection by spike-pseudotyped lentiviral particles.

### Chewing Gum for Prevention of Transmission of Influenza

Since the first influenza pandemic in 1918, and 2005/2005 epidemic, WHO has set up 149 national centers for monitoring influenza. Influenza in humans is an infectious respiratory disease. There are four types of influenza viruses: A (IAV), B (IVB), C (ICV) and D (IDV). A and B types are the most common, and type A is responsible for pandemics and seasonal epidemics. Influenza A, B viruses have eight linear segments of single stranded RNA with a diameter of 80-120 nm and mass about 170-200,000 kDa (Paules & Subbarao, Lancet 2017, 390, 697-708; Jang & Seong, 2017 Front. Cell Infect. Microbiol. 9, 344). Almost 40% of the flu virion surface is covered by the spike protein, with 350 spikes of hemagglutinin (H subtype) and 70 spikes of neuraminidase (N subtype) and mutations affect infectivity of viral particles (Ksenofontov et al. Molekulyarnaya Biologiya, 2008, Vol. 42, No. 6, pp. 1078-1080). The avian influenza virus (H7N9) epidemic occurred in China in 2013 and 2017. Influenza viruses use sialic acid receptors in host cells with the receptor binding domain of the HA protein. New flu vaccines are developed for each seasonal flu due to antigenically drifted variants (Krammer et al. Nat. Rev. Drug Discov. 14 (3) (2015) 167-182). While vaccines are developed against IAV, novel evolving avian/mammalian or bat derived serotypes (H1-H18, N1-N11, Wu et al. Trends Microbiol. 22 (4) (2014) 183-191) pose greater danger. Using the concept described in this example for the SARS-Cov-2 spike protein, influenza virus transmission during seasonal flu could be decreased by debulking the virus with engineered IAV blocking peptides (or FRIL, as disclosed in Example 2) in chewing gums. Such peptides could include portions of the HA and or neuraminidase proteins. These compositions and methods will provide therapeutic and public benefit by reducing viral load in affected subjects.

### Example II

### Debulking Different Corona (SARS-CoV-2 Delta, Omicron, OC43) and Influenza (H1N1, H3N2) Virus Strains in the Oral Cavity by Trapping Molecules in Chewing Gums to Decrease Infection and Transmission

Oral diseases caused by microbial infections afflict 3.5 billion people worldwide. Bacteria and fungi colonize tooth surfaces forming tenacious and intractable biofilms resulting in severe dental caries, while saliva is a major source of pathogens that are transmitted as droplets or aerosolized particles [1]. Of recent concern is COVID-19, in which the salivary glands are the primary replication site of SARS-CoV-2, leading to the loss of taste and smell [2-5]. In addition, Influenza, HPV, HSV1, EBV and KSHV viruses are also transmitted orally and their life cycle in the oral epithelium is well characterized [6-11]. Each of these viruses possess glucans which can be simple or complex on their surfaces which can be bound by FRIL.

Although SARS-CoV-2 transmission between unvaccinated individuals is the primary cause of continued spread, fully vaccinated individuals with breakthrough infections have peak viral loads similar to unvaccinated individuals and efficiently transmit virus in household settings [12]. Equally important is the pattern of SARS-CoV-2 evolution that strengthen viral infectivity through antibody-resistant mutations [13]. Although SARS-CoV-2 is transmitted nasally and orally, oral transmission is 3-5 orders of magnitude higher than nasal transmission [14-28]. Airborne-lifetime-weighed volume of saliva droplets in healthy subjects is 3-5 orders of magnitude higher than breath droplets; speaking four words releases more virus particles than an entire hour of breathing, suggesting that a decrease in oral viral load could have substantial effect on virus transmission [14-28]. Therefore, new methods are proposed to debulk pathogens in the oral cavity and minimize transmission.

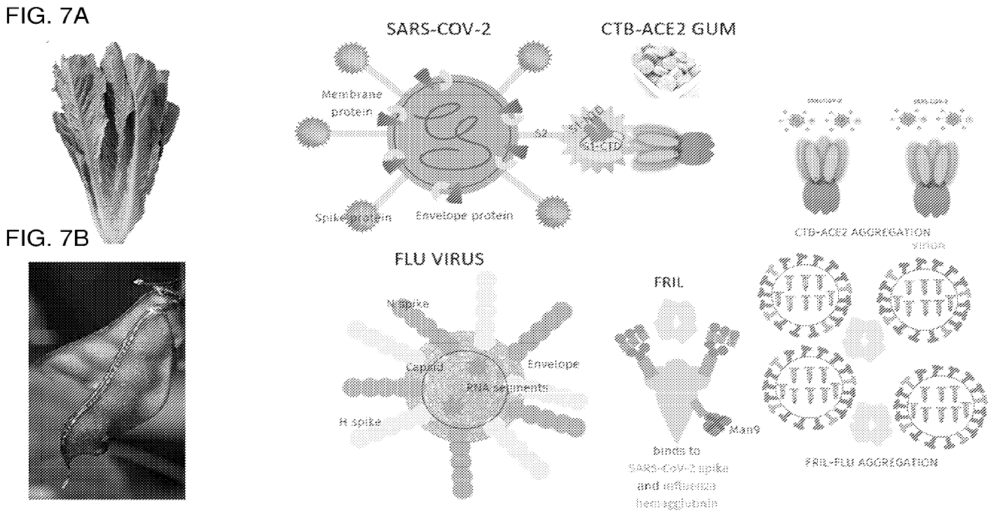

Clinical evaluation of mouth rinses in COVID-19 patients reveal no statistically significant changes in saliva viral load after rinse, up to two hours [29]. It is possible that qPCR detects non-viable viral particles, as evidenced by detection of SARS-CoV-2 several weeks after disappearance of symptoms [30-32] and subsequent CDC guidelines to not perform qPCR testing up to ninety days after the onset of infection. ACE2 enzyme has been expressed in chloroplasts to treat pulmonary hypertension in previous studies, which is now advancing to the clinic to treat COVID-19 patients. In addition, as explained above in Example I. CTB-ACE2 chewing gum was able to markedly debulk SARS-CoV-2 (>95%) in COVID-19 patient saliva or swab samples as measured by microbubbles or qPCR [34]by direct binding of the spike protein to soluble ACE2 (FIG. 7A). Blocking engagement of spike protein to ACE2 and GM1 receptors by CTB-ACE2 (FIG. 7A) was evaluated by inhibiting uptake of lentivirus or VSV pseudo-type virions into Vero cells. In addition to the native human ACE2 enzyme used in this study, several mutated versions with much higher affinity to the SARS-CoV-2 have been developed and could also be utilized as viral trap proteins [35,36].

The foremost viruses with the strongest capabilities for spread by aerosol transmission and causing illness and death to the widest population are SARS-CoV-2 and Influenza. The significance of the plant lectin FRIL is its preferential entrapment of viruses that express complex-type N-glycans (FIG. 7B) on their outer envelopes [38,39]. Of note enveloped viruses express either high mannose, complex N-linked glycans, or a non-complex-hybrid polysaccharides. Both SARS-CoV-2 and Influenza (Flu) viruses have been shown to contain complex-type N-glycans on their envelopes [38-41]. Influenza (Flu) is an especially significant respiratory virus that affects the worldwide population [42]. The infections are seasonal and mainly endemic with a high rate of morbidity and mortality [42]. Globally, influenza causes half a million deaths annually [40]. Each year a new Flu vaccine is deployed based on emerging strains with small and gradual variations (Genetic Drift) [42]. Flu is especially life-threatening when the segmented genomic strands that separately encode HA and NA of human Flu become mixed upon infection with a different species e.g., bird or pig (Genetic Shift) [42]. Several major pandemics have occurred in the past 150 years with 1918 being the worst case, resulting in 50 million deaths and infecting one-third of the world's population [40,42]. A potential global outbreak of Avian H5N1 Bird Flu appeared in China in 2013; the Bird Flu directly infected humans with the potential to spread worldwide [43].

Based on the success of the COVID gum to debulk SARS-CoV-2 using the native human protein ACE2. (see Example I), in this example, we explored the entrapment efficacy in different strains of SARS-CoV-2 using microbubbling (N-antigen) or RAPID (spike protein) assays. In addition, we investigated the virus trap plant protein lectin (FRIL) for its potential to neutralize SARS-CoV-2 and influenza virus by plaque reduction assay and mechanism of entrapment using electron micrography. For five decades protein drugs have been delivered as sterile injections, requiring cold storage/transportation, thereby decreasing patient affordability and compliance. In order to address some of these challenges, the Daniell lab has developed oral delivery systems through encapsulation of protein drugs in plant cells [44-49]. Plant cell bioencapsulation eliminates cold storage/transportation challenges [50-53]. Plant cells are now being developed as a novel strategy to deliver protein drugs against pathogens that colonize the oral cavity by disrupting biofilm to kill pathogens that cause dental caries [50] or debulking SARS-CoV-2 in saliva to decrease reinfection and transmission [34]. This approach is especially suitable for reducing viral load in saliva or clearing the throat surface, where most viral infections originate. While chewing gums have been used since 1928 [54] to deliver small molecules like aspirin [55] caffeine [56], calcium carbonate [57], chlorhexidine [58], nicotine [59,60], and Xylitol [61], delivering protein via gums pose additional challenges in their stability and release kinetics. For example, insulin in chewing gum tablets was mostly degraded in the gastric juice and was not validated by animal testing [62] but oral delivery of insulin bioencapsulated in plant cells is feasible [63]. Similarly, when proteins are bioencapsulated in plant cells, they are not degraded during the gum manufacturing process (requiring high temperature) and are stable for several years in chewing gum [34,50]. In this study, we optimize protein release kinetics from the chewing gum to initiate clinical trials of proteins in the oral cavity.

### Materials and Methods for Example II

### Preparation of Medical Grade CTB-ACE2 Lettuce Drug Protein for Manufacturing of Chewing Gum Tablets

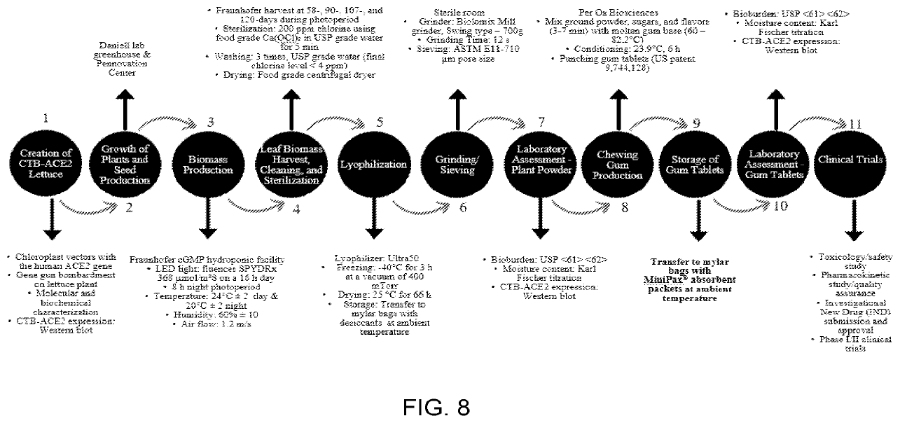

CTB-ACE2 lettuce plant material was grown, washed and lyophilized at Fraunhofer according to the procedure described previously in Example I. Grinding of plant material was performed on a pre-disinfected bench in a clean room facility using a steel grinder (BioloMix Mill Grinder, Swing-700g) which was washed and rinsed thoroughly to remove any traces of detergent on the surface. All washed equipment's were disinfected using 70% Isopropyl Alcohol (IPA)/Ethanol to remove any bio-load attached to the surface. Forceps, aluminum foil sheets and sieve (USA standard sieve-ASTM E11 specifications; No. 25; 710 μm) were autoclaved at 121° C. for 20 min. The lyophilized leaves were placed on the sterile aluminum foil sheet and placed on the clean bench and mid-ribs were removed using the pre-sterilized forceps. Ten grams of the plant material was weighed on a pre-disinfected weighing scale and transferred to the grinder. Plant material was ground for 12 seconds. Time for grinding was carefully monitored using Traceable Nano Timer (Fisher Scientific). The milled powder was aseptically sieved through onto sterile aluminum foil using ASTM E11 No. 25 710 μm pore size sieve and transferred to presterilized Uline black container. All leftover materials on the sieve were discarded. The Uline containers containing material were stored in a steel cabinet at room temperature. An aliquot of 100 mg ground sample was aseptically removed in sterile container for bioburden assessment to evaluate total microbial and yeast/mold counts as per USP <61> and <62>. Moisture content of the plant material used for gum preparation was determined by the protocol described above (FIG. 8).

### Preparation of CTB-ACE2 Chewing Gums

Chewing gum tablets containing ground CTB-ACE2 plant powder were produced by Per Os Biosciences (Hunt Valley, MD) by a compression process that preserves the efficacy of the active ingredient instead of the traditional gum manufacturing technique which routinely involves extrusion/rolling at high temperatures that can introduce variability in the protein concentration and degrade its efficacy. The CTB-ACE2 gum tablets were prepared with the following excipients—gum basce (24.46%), magnesium stearate (3.00%), maltitol (15.98%), Xylitol (1.98%), sorbitol (20.93%), silicon dioxide (0.40%), isomalt (10.00%), stevia 99% (0.45%), natural flavoring agents (maltodextrin, dextrose, gum arabic, essential oils) to make the gum tablets flavorful, and conducive to compression. The gums thus manufactured containing 50 mg plant powder (2 g weight/tablet) performs exactly like conventional gums available in the market with respect to physical characteristics. The gum tablets received from Per Os Biosciences were stored in the mylar bags to avoid moisture absorption. Few tablets were kept in the Uline black containers for routine examination viz; bioburden, moisture content, drug dose quantitation and release (FIG. 8).

### Total Dose and Release Quantification of CTB-ACE2 Gum

The total dose of CTB-ACE2 in the 2 g gum tablet was examined by western blot technique. 100 mg of the crushed gum powder was suspended in 500 μL of plant extraction buffer (100 mM NaCl; 10 mM EDTA; 200 mM Tris-HCl. pH 8.0; 0.05% (v/v) Tween-20; 1× protease inhibitor cocktail; 0.1% SDS; 14 mM ß-Mercapto-ethanol; 400 mM sucrose; and 2 mM Phenyl-methylsulfonyl fluoride (PMSF)) and incubated for 1 h at 4° C. on a vortex. This was followed by sonication for 6 cycles at 80% amplitude for 10 s on and 15 s off using a sonicator 3000 (Misonix, Farmingdale, NY). Bradford assay for total protein quantitation followed by immunoblot analysis for total CTB-ACE2 dose quantitation were performed following protocols developed by Daniell lab. See Example I.

For evaluation of the CTB-ACE2 release, 100 mg of the crushed gum tablet was suspended in 500 μL of plant extraction buffer (PEB) (10 mM EDTA; 400 mM Sucrose; 100 mM NaCl: 0.05% (v/v) Tween-20; 0.1% SDS; 14 mM ß-Mercapto-ethanol; 200 mM Tris-HCl, pH 8.0; 2 mM PMSF; and 1× protease inhibitor cocktail) and incubated for 30 min while vortexing at 4° C. This was followed by centrifugation at 750 g for 5 min at 4° C. The supernatant fraction was stored on ice until analysis. The remaining pellet fraction was resuspended in PEB and sonicated for 3 cycles at 80% amplitude for 5 s on and 10 s off using a sonicator 3000 (Misonix, Farmingdale, NY). Bradford assay for total protein quantitation followed by immunoblot analysis for CTB-ACE2 release were performed following protocols developed by Daniell lab.

### Nasopharyngeal Patient Swab Sample Preparation

Oropharyngeal (OP) and nasopharyngeal (NP) swabs samples were collected from patients admitted to the Hospital of the University of Pennsylvania with clinically-confirmed COVID-19 infection using flocked nylon swabs (Copan Diagnostics). The OP and NP swabs were eluted together in 1.5 ml of viral transport media (VTM) as previously described [64], aliquoted and frozen (−80° C.) prior to analysis. Informed consent was provided by all study participants under protocols approved by the University of Pennsylvania IRB (protocol #823392). Virus quantification was carried out by qPCR using N1 primers as previously described [64]. Virus lineage assignment (Table 2) was based on whole genome sequencing and assignment using Pangolin lineage as described [65], or based on S gene target failure in the patient's clinical diagnostic sample RT-PCR, which is a marker for the Omicron lineage [66, 67]; in some cases Omicron lineage assignment was based on near-100% Omicron circulation within the local community at time of sampling.

TABLE 2
Viral titer information on Omicron variant of SARS-CoV-2 NP/OP
samples treated with FRIL and CTB-ACE2 antiviral trap proteins
and assessed by microbubble SARS-CoV-antigen assay and RAPID.

| | | Virus titer | | | |
|---|---|---|---|---|---|
| | | per μL | | | |
| | Actual | RNA (N1 | Virus titer per μL | Variant of | Assignment |
| Sample | sample type | qPCR) | neat sample * | Concern | method |
| 620 | NP/OP | 8.33E+02 | 2.98E+02 | Omicron | WGS |
| 613 | NP/OP | 2.78E+04 | 9.93E+03 | Omicron | WGS |
| 614 | NP/OP | 1.40E+01 | 5.00E+00 | Omicron | SGTF |
| 615 | NP/OP | 1.17E+01 | 4.18E+00 | Omicron | SGTF |
| 593 | NP/OP | 8.70E+02 | 3.11E+02 | No | WGS |
| 595 | NP/OP | 9.63E+02 | 3.44E+02 | Delta | WGS |
| 596 | NP/OP | 8.84E+03 | 3.16E+03 | Delta | WGS |
| 597 | NP/OP | 5.30E+04 | 1.89E+04 | Delta | WGS |
| 600 | NP/OP | 2.59E+05 | 9.25E+04 | Delta | WGS |
| 601 | NP/OP | 1.19E+04 | 4.25E+03 | Delta | WGS |
| 603 | NP/OP | NA | NA | Delta | Date |
| 605 | NP/OP | NA | NA | Delta | Date |
| 607 | NP/OP | NA | NA | Delta | Date |
| 609 | NP/OP | 5.17E+03 | 1.85E+03 | Delta | WGS |
| 613 | NP/OP | 2.78E+04 | 9.93E+03 | Omicron | WGS |
| 615 | NP/OP | 1.17E+01 | 4.18E+00 | Omicron | SGTF |
| 619 | NP/OP | 1.02E+01 | 3.64E+00 | Omicron | SGTF |
| 620 | NP/OP | 8.33E+02 | 2.98E+02 | Omicron | WGS |
| 624 | ETA | 6.22E+01 | 2.22E+01 | Omicron | SGTF |
| 626 | ETA | 1.55E+03 | 5.54E+02 | Omicron | WGS |
| 628 | NP/OP | NA | NA | Omicron | Date |
| 629 | NP/OP | NA | NA | Omicron | Date |
| 630 | NP/OP | NA | NA | Omicron | Date |
| 631 | NP/OP | NA | NA | Omicron | Date |
| 476 | NP/OP | 5.92E+05 | 2.11E+05 | Unknown | |
| 482 | NP/OP | 6.32E+06 | 2.26E+06 | Unknown | |
| 505 | NP/OP | 1.17E+06 | 4.18E+05 | Unknown | |

WGS—whole genome sequencing (Polar Protocol and ARTIC primers)
SGTF—S gene target failure by PCR, presumed Omicron
Date—circulation at time of complete or near-complete variant dominance
* Adjusting for the fact that RNA from 140 μl of neat sample was eluted in 50 μL buffer
NA: not available

### Microbubbling SARS-CoV-2 Antigen Assay

The microbubbling SARS-CoV-2 antigen assay was performed with the clinical NP swab samples as described above. Out of the available four patient NP/OP samples of the omicron variant of SARS-CoV-2, two were tested with the FRIL bean powder and the remaining with CTB-ACE2 gum powder. Briefly, patient samples (150 μL) were incubated on the vortex machine with different doses of FRIL bean powder (5, 10, 25 mg containing 20, 40, 100 μg protein respectively) and CTB-ACE2 gum (10, 25, 50 mg containing 0.18, 0.46, 0.92 μg respectively) at 4° C. for 30 minutes. This was followed by centrifugation at 14000 rpm for 20 minutes at 4° C. The supernatant thus recovered (120 μL) was carefully collected in separate tubes without disrupting the pellet. The supernatant was first treated with the lysis buffer of 10% Tween 20 (100×, 1.2 μL) and 100× protease inhibitor cocktail (1.2 μL) and incubated for 30 min at room temperature. 100 μL of the lysed samples were incubated with suspensions of 500,000 capture antibody functionalized magnetic beads in a 96 well-plate and secured on a rotator (12 rpm) for 30 min at room temperature. The 96-well plate was then placed on a magnet which separates the magnetic beads and the wells were washed thrice using washing buffer (0.05% Tween 20 in PBS buffer, pH 7.4) followed by resuspension in 100 μL of 250 ng/mL biotinylated detection antibody in PBS containing 1% BSA. After incubation for 30 min at room temperature, the beads were similarly washed thrice followed by resuspension in 100 μL of 1 μg/mL NeutrAvidin functionalized Pt nanoparticles at room temperature for 30 min. The beads were then washed thrice and finally resuspended in 100 μL of 30% H₂O₂. The magnetic bead mix containing immunosandwich complexes formed between magnetic beads/target protein/PtNps were transferred to the previously designed [68,69] microbubbling microchips containing microwell arrays (14 μm×14 μm, 7 μm depth, 100×100). These microchips were then placed on a neodymium disk magnet subjecting it to an external magnetic field for 9 min to pull down the beads to the microwells. Microbubbles formed as a result of the accumulation of oxygen in the microwells catalyzed by PtNPs through H₂O₂ decomposition were imaged using an iPad with the uHandy mobile phone microscope (9× magnification, 5 mm focusing length; Aidmics Biotechnology, Taipei, Taiwan).

### Rapid Assay

The electrochemical sensors were prepared as described previously [70]. A SquidStat Plus (Admiral Instruments) potentiostat and Electrochemical impedance spectroscopy (EIS) were used for viral detection. The EIS measurements were performed as described previously [70]. NP/OP patient swab samples of SARS-CoV-2 delta and omicron variants were heat inactivated for 1 h at 56° C. For RAPID assays, 150 μL of each sample was treated with 20 mg of CTB-ACE2 crushed gum powder for 1 hour under stirring at 4° C. After incubation, samples were vortexed again and spun down at 14,000 rpm for 20 min at 4° C. First, 10 μL of VTM (blank) were added to the working electrode and left for 2 minutes, followed by addition of 200 μL redox probe to cover all the electrodes (counter, reference, and working electrodes), and EIS experiments were performed to obtain the blank signal. After the blank was analyzed, the sensor was washed with PBS, pH 7.4 and 10 μL aliquots of the resulting supernatant of the samples before and after incubation and centrifugation of the CTB-ACE2 chewing gum were placed directly onto the working electrode of the biosensor. The sample was removed after 2 minutes of exposure, cleaned carefully with PBS and after addition of 200 μL of the redox probe EIS analyses were performed.

### Cell Lines

African green monkey kidney epithelial Vero E6 cells were cultured in Dulbecco's modified Eagle's medium (DMEM, Invitrogen), supplemented with 5% heat-treated fetal bovine serum (FBS, Sigma), 100 units/mL penicillin, 2 mM L-glutamine, 50 μg/mL gentamicin, 100 μg/mL streptomycin, 1.25 μg/mL of amphotericin B (Fungizone), and 10 mM HEPES (4-(2-hydroxyethyl)-1-piperazine ethanesulfonic acid, pH 7.2). Cells were incubated with 5% CO² at 37° C. Madin-Darby Canine Kidney (MDCK) cells were cultured in Minimum Essential Media Alpha (MEM α, Gibco), supplemented with 5% heat-treated fetal bovine serum (FBS, Sigma). 100 units/mL penicillin, 2 mM L-glutamine, 50 μg/mL gentamicin, 100 μg/mL streptomycin, and 1.25 μg/mL of amphotericin B (Fungizone), and 10 mM HEPES (4-(2-hydroxyethyl)-1-piperazine ethanesulfonic acid, pH 7.2).

### FRIL Protein Purification

Lablab purpureus bean powder was extracted in PBS buffer and dialyzed with reduced levels of salt concentrations overnight. Next, the sediment was resuspended in 20 mM phosphate buffer (pH 8.0) and transferred onto an Unosphare Q column (BioRad, Hercules, California). Bonded proteins were extracted with different gradients of NaCl (0 to 0.5M). The fractions with the highest neutralization titer were pooled, concentrated, and loaded onto a Superdex s200 10/300GL size exclusion column (GE, Boston, Massachusetts). The fractions with the highest neutralization titers were then pooled and concentrated. Finally, the bands representing FRIL were isolated through cibardon blue affinity chromatography (Affi-Gel, BioRad) flow-through and separated from nonspecific bands at ˜30 and 40 kDa.

### Plaque Reduction Assay

Influenza Virus: Purified FRIL as well as lablab bean powder, as the source of FRIL, were evaluated for their abilities to prevent infection of influenza virus strains H1N1 (A/California/7/2009-X181) and H3N2 (A/Singapore/INFMH-16/0019/2016) using a quantitative viral plaque reduction assay. The assay was conducted in 100 μL by co-incubating the Flu strains (80 pfu) with increasing amounts of purified FRIL (0 to 3.2 μg) in serum-free medium or protein extract of Bean Powder (0 to 2 mg) in PBS at 37° C. for 1 hour. The virus plus FRIL and powder, respectively, were then added onto MDCK cells (at ˜90% confluence) in 48-well plates for infection. Following 1 h adsorption at 37° C., the virus mixtures were aspirated and washed to eliminate unabsorbed Flu. The cells were overlaid with Avicel/methycellulose, incubated at 37° C. for 28 h, fixed and immune-stained with anti-Flu nucleoprotein antibody. Viral plaques were microscopically counted and used to generate dose response curves.

Coronavirus: OC43 was co-incubated with increasing amounts of purified FRIL (0 to 3.2 μg) in serum-free medium or protein extract of lablab bean powder (0 to 2 mg) in PBS for 1 hr. Vero cells were then infected by adsorbing 100 μL of the OC43 FRIL mixtures at 34° C. for 1 hr in serum free medium and then placed in culture medium containing heat-treated serum for 5 days at 37° C. after which cells were fixed and stained in 4% formaldehyde and 0.2% crystal violet. Viral plaques were microscopically counted and used to generate dose response curves.

### Negative Staining Electron Microscopy

H1N1 virus culture: H1N1 (A/California/7/2009-X181) viruses and purified FRIL protein (10 μg/mL and 150 μg/mL) were co-incubated in HEPES buffer (50 nM, pH=8.0) at 37° C. for 60 minutes. The H1N1 virus was diluted from a stock concentration of 4×10⁷ pfu/mL using HEPES buffer to a titer of 1×10⁶ pfu/mL. The virus and purified FRIL protein were pretreated with centrifugation at 15,000 rpm for 5 minutes. After incubation, samples were applied to a glow-discharged carbon-coated 400 mesh copper grid. The carbon-coated grid is stained with 2% uranyl acetate and washed twice with 5 μL of diH₂O. The viruses were then observed using transmission electron microscope (FEI Tecnai T12) operating at 100 kV with a CMOS camera (Gatan Oneview, Pleasanton, California). Images were captured at the magnification of 42K using Gatan Digital Gatan Digital Micrographic software.

Purified H1N1 virus: Sucrose-gradient purified viruses were used for microscopic visualization. H1N1 (A/California/7/2009-X181) viruses and 150 μg/mL of FRIL were co-incubated at 37° C. for 30 minutes. Glow-discharge treatment of the carbon-coated 400 mesh copper grid was performed using PELCO casiGlow™ 91000 Glow discharge cleaning system (Ted Pella Inc., USA). The grids were glow discharged with 15 mA current for 30 seconds. After incubation, samples were diluted with PBS buffer and loaded onto the carbon-coated grid. The grid is negatively stained with Nano-W (Nanoprobes, Yaphank, New York). The viruses were observed using electron microscope (JEM-1400. JEOL, Peabody, Massachusetts) operating at 120 kV with a CCD camera (Gatan 895. Gatan, Pleasanton, California). Images were captured at the magnifications of 2.6K and 5K by Gatan Digital Micrographic software.

### Statistical Analyses

CTB-ACE2 total dose quantitation and release data are presented by means±SD. Microbubbling SARS-CoV-2 Antigen Assay data are presented by means±SD. Statistical significance was determined using by One-Way Electrochemical impedimetric measurements are presented as an average of 3 replicates for each condition. Graphs were created and statistical tests were conducted in GraphPad Prism 9.2. In plaque reduction assay, the plaque numbers were quantified under a dissecting microscope. Half-maximal EC50 values were obtained by nonlinear regression fitting to a variable slope, four parameter dose-response model using the Prizm 6 software (GraphPad Software, LaJolla, CA).

### Results

As pointed out in the introduction, oral transmission of SARS-CoV-2 is 3-5 orders of magnitude higher than nasal transmission [14-28]. After two years of the current pandemic, there is still no FDA approved quantitative detection method to end the quarantine or return of employees back to workplaces after testing positive for SARS-CoV-2. It is possible that qPCR detects non-viable viral particles, as evidenced by detection of SARS-CoV-2 several weeks after disappearance of symptoms [30-32] and CDC guidelines do not support qPCR testing up to ninety days after the onset of infection. In order to develop a quantitative antigen test and study the changes in viral load in COVID-19 samples in response to viral trap proteins, this study utilizes two different approaches. Microbubbling (N antigen) and RAPID (spike protein) assays are used to evaluate CTB-ACE2 gum or the lablab bean powder containing the viral trap protein—FRIL (FIG. 7A, 7B). In addition, the impact of FRIL on influenza virus strains is evaluated in plaque reduction assays. Mechanism of FRIL entrapment is further investigated using electron microscopic studies. Various steps involved in the process of creation of plants expressing CTB-ACE2, production of seeds, biomass, sterilization, lyophilization, grinding/sieving, production of chewing gum with lyophilized plant powder, evaluation of FDA requirements (moisture content, bioburden, drug dosage, stability, etc.), functional characterization, toxicology/pharmacokinetic studies, filing of IND and clinical trial design are summarized in FIG. 8.

### CTB-ACE2 Dose Quantitation and Release from Gum Tablets During Incubation

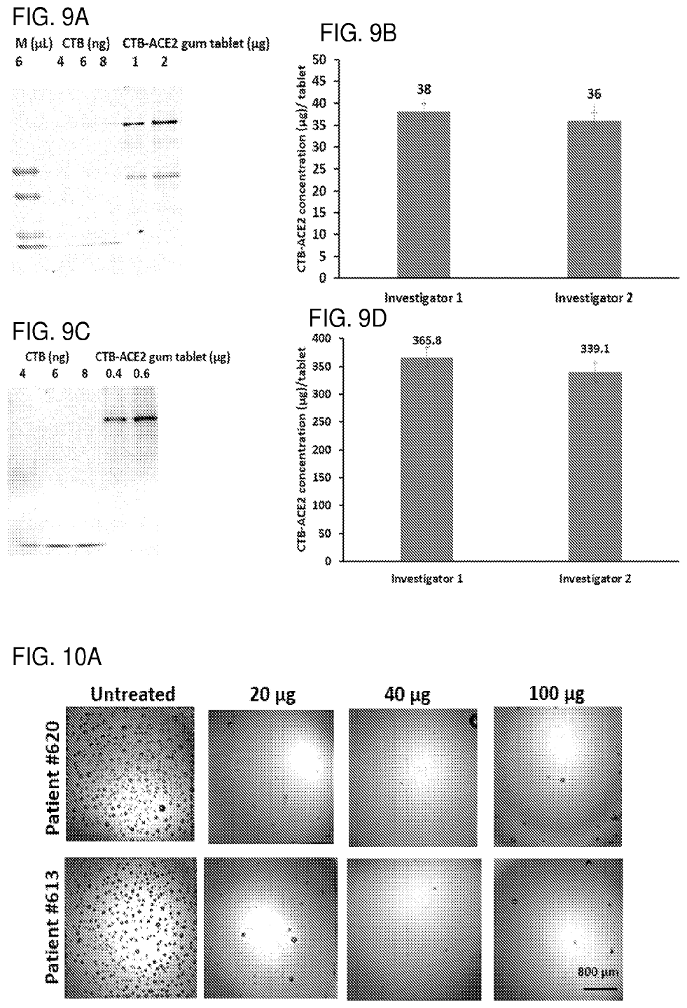

Western Blot Assessment of Gum Tablet Revealed the Total Dose of CTB-ACE2 to be 352.45±18.9 μg/2 g tablet (FIG. 9A, 9B). CTB-ACE2 release without sonication was found to be 37±1.0 μg/tablet (2 g) (FIG. 9C, 9D) under optimized experimental conditions performed in the lab. The grinding conditions were optimized to maximize the release of protein conducive for chewing gum drug delivery system. This is different from the orally delivered protein drugs [49,63] that are bioencapsulated and hence protected from degradation in the stomach by gastric juices and subsequently released in the gut by microbial digestion of the plant cell wall.

Bioburden assessment for plant material used for preparation of gum and the gum tablets revealed no microbial or fungal growth adhering compliance to the FDA parameters. Moisture content of clinical grade CTB-ACE2 lettuce powder was found to be 5.5% which meets the FDA requirements for orally delivered plant powder. The gum tablets prepared thus, comply for the FDA specifications and ready to be used for clinical trials.

### Evaluation of SARS-CoV-2 in Clinical Swab Samples by Microbubbling after Treatment with CTB-ACE2 Gum and FRIL Bean Protein

The microbubbling SARS-CoV-2 antigen assay is designed to detect the nucleocapsid (N) antigen at femtomolar concentrations [68]. Due to limited sample volume availability, dose dependent analysis was restricted to three doses. Omicron variant NP samples from patients #620 and #613 were tested for the debulking potential of FRIL bean protein at doses 20, 40 and 100 μg each and patients #614 and #615 were tested for the same with CTB-ACE2 gum at doses 0.18, 0.46 and 0.92 μg. The viral titers for all four omicron variant NP samples are shown in Table 1.

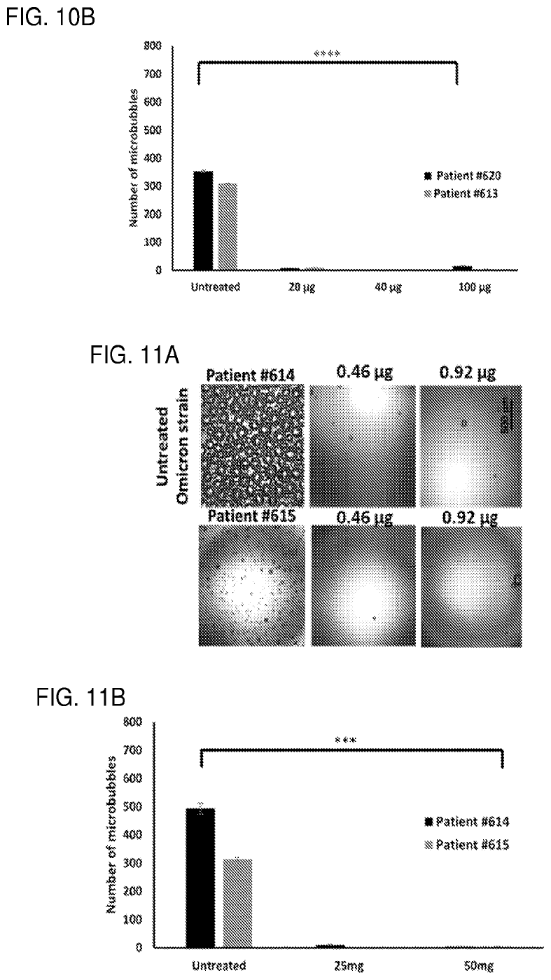

As seen in FIGS. 10A, and 10B FRIL protein is capable of significantly reducing the microbubble count (p<0.0001) even at the lowest dose of 20 μg.

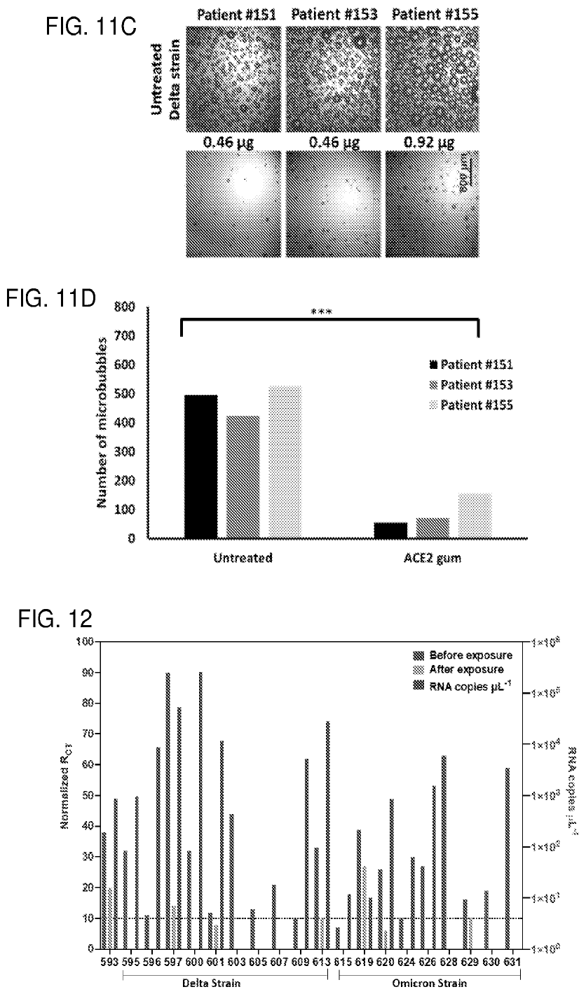

A dose dependent reduction in microbubbles was seen with CTB-ACE2 gum, with least number of microbubbles seen in the 0.92 g treated sample (p=0.0001) (FIG. 11A, 11B).

As we compare the doses between both plant-based anti-viral trap proteins, it is interesting to note that CTB-ACE2 is effective at a dose of 0.46 μg while almost similar debulking potential was seen with 20 μg of FRIL. In our recently published paper we have shown the debulking efficacy of CTB-ACE2 against the delta variant of the virus as seen in FIG. 11C, 11D. The neutralization potential of CTB-ACE2 is effective across both variants of the SARS-CoV-2. These results also confirm that the SARS-CoV-2 debulking efficiency of FRIL, a plant lectin, is distinct from that of CTB-ACE2. While ACE2 binds directly to the SARS-CoV-2 spike protein and entrapping the virus in the pentameric insoluble microparticle structure of CTB-ACE2 [34], FRIL preferentially binds to complex-type-N-glycans on viral glycoproteins forming aggregates [38]. Both these distinct mechanisms facilitate removal of entrapped viral particles and hence could be an effective preventive measure in limiting viral transmission. In all four patient swab samples, we do not observe a positive correlation between the viral load titer as determined by RT-PCR and the microbubble count that depends on the presence of N antigen (Table 1). Patients (#620, #613) with higher viral load have lower microbubble count, which suggests that RT-PCR may be detecting N1 gene fragments that do not produce intact N1 antigens with epitopes for antibody binding [69].

### Evaluation of SARS-CoV-2 in Clinical Swab Samples by RAPID after Treatment with CTB-ACE2 Gum

Our previously developed method RAPID (Real-time Accurate Portable Impedimetric Detection) [70,71], which uses electrochemical impedance spectroscopy (EIS) to detect the binding between spike protein and human receptor ACE2, was used here to validate the trapping effect of the CTB-ACE2 chewing gum [72-76]. Based on our prior work with hundreds of clinical samples in order to elucidate the optimal analytical conditions for the assay [70,71], we used the normalized Rer response, defined by the following equation:

$$normalized {R}_{C T} = \frac{Z - {Z}_{0}}{{Z}_{0}}$$

where Z is the R_{CT} of the sample group and Z₀ is the R_{CT} of the blank group (virus transportation medium, VTM). RAPID presents low limits of detection (LOD, 6.29 fg mL⁻¹ SP) and quantification (LOQ, 20.96 fg mL⁻¹ SP) in VTM medium based on the signal to noise ratio (S/N=3) and (S/N=10), respectively [70]. With the exception of one sample (ID 593), the samples were genotyped based on whole genome sequencing or S gene target failure or date of collection (Table 1, FIG. 12). Samples 595-609 are Delta and 613-631 are Omicron strains of SARS-CoV-2. RNA extraction for all samples were from equal patient sample volume (140 μl) and RNA extracted was resuspended and quantified by qPCR under similar conditions. There is no unique change observed in viral load between Delta and Omicron samples, but viral load varied significantly among patients. Patient samples (150 μl of sample) were incubated with the CTB-ACE2 chewing gum and RAPID was performed in 10 μl aliquots of the patient sample (FIG. 12) and R_{CT} values varies significantly among patient samples. The CTB-ACE2 chewing gum was able to trap viral particles efficiently, decreasing viral concentration in all the 20 samples analyzed (FIG. 12). For 17 of the samples, no viral particles were detected by RAPID after the incubation with the CTB-ACE2 chewing gum, underscoring the ability of this material to trap the viral particles up to very low SARS-CoV-2 SP concentrations levels (below the LOD of RAPID).

### FRIL Exhibits Potent Neutralization of Influenza Virus and Coronavirus

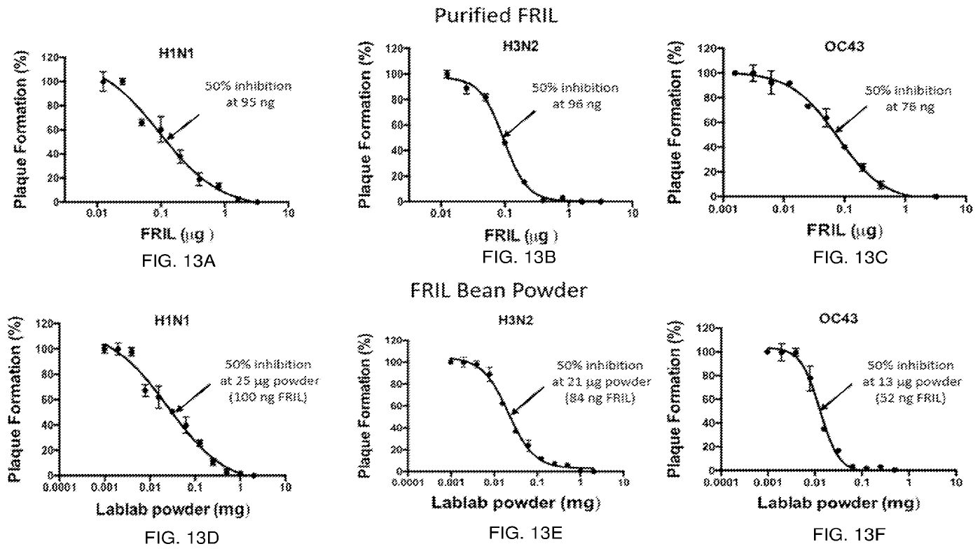

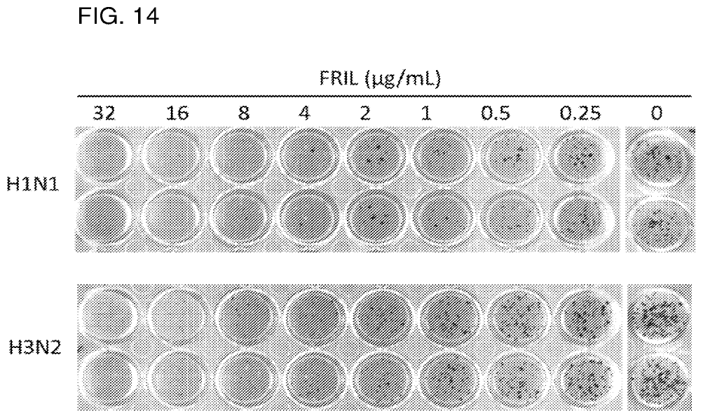

As FRIL has binding affinity to complex-type N-linked glycans, it could be effective against a wide spectrum of enveloped viruses that express complex-type N-glycans, such as Influenza virus, HBV, and HSV [79-81]. Here, we examined the antiviral activities of purified FRIL protein and lablab bean powder against the Influenza H1N1 (A/California/7/2009-X181), and H3N2 (A/Singapore/INFMH-16/0019/2016), as well as Coronavirus HCoV-OC43 using plaque reduction assays (FIG. 13, FIG. 14).

In the Influenza plaque reduction assays, purified FRIL protein exhibited a midpoint inhibition at 95 ng (in 100 μL treatment volume) against H1N1 (FIG. 13A) and 96 ng against H3N2 (FIG. 13B). Interestingly, FRIL's antiviral potency of 76 ng against OC43 was experimentally close to that of the Influenza viruses (FIG. 13C), taking into consideration possible minor differences in viral titers and plaque counts. These results correspond to the previously reported 50% plaque reduction neutralization test (PRNT₅₀) value against H1N1, H3N2, and hCoV-19/Taiwan/NTU04/2020 [38].

Importantly, the anti-viral capacity of lablab bean powder has never been studied. Compared to purified FRIL, lablab bean powder is a better candidate for anti-viral chewing gum with advantages related to affordability, accessibility, and stability of enzymes. Therefore, we examined the neutralization ability of lablab bean powder, which contains 4 μg FRIL protein per 1 mg of bean powder. The concentration of total soluble lablab protein obtained was 149.28 μg per 1 mg of starting lablab bean powder. Plaque assays with soluble lablab bean powder exhibited mid-point inhibition values of 25 μg (estimated to release 100 ng FRIL) against H1N1 (FIG. 13D), and 21 μg (84 ng FRIL) against H3N2 (FIG. 13E) and 13 μg (52 ng FRIL) against OC43 (FIG. 13F). More importantly, the estimated amount of solubilized FRIL released at midpoint inhibition is experimentally close to the amount of purified FRIL used for midpoint inhibition, taking into consideration possible minor differences in viral titers and plaque counts. For example, the lablab bean powder reaches mid-point inhibition at 25 μg for H1N1 which is estimated to release 3732.5 ng solubilized protein and 100 ng solubilized FRIL, while purified FRIL reaches midpoint inhibition at 95 ng solubilized FRIL. This is significant in that the antiviral activity is via solubilized FRIL, while lablab bean powder contains 370-fold more of other plant proteins. These data show that purification of FRIL from lablab bean powder is not required for its antiviral capacity, which lays the logical foundation for manufacturing chewing gum with lablab bean powder.

In summary, for both Influenza stains and the OC43 Coronavirus, less than 1 mg/ml of lablab bean powder (which releases 4 μg/mL of FRIL) can effectively inhibit all infections. Considering the volume of saliva in the mouth before swallowing is 0.87 ml in males and 0.66 ml in females [81], we estimate that <1 mg of lablab bean powder in chewing gum could effectively debulk different Influenza virus strains and Coronavirus in saliva and thereby prevent infection. Such high potency of FRIL bean powder allows for the manufacturing of chewing gum containing FRIL that could effectively reduce the transmission risk of both Influenza and Coronavirus.

### Mechanism of Virus Entrapment by FRIL Bean Protein

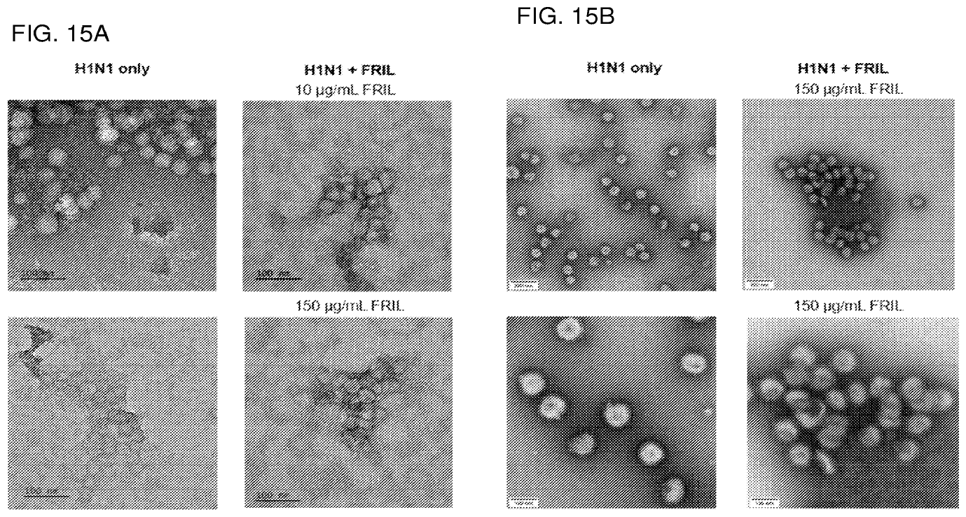

Different from the debulking mechanism of CTB-ACE2. FRIL entraps virions through its binding affinity to the complex-type N-linked glycans on virus envelopes. Assemblies of entrapped virus particles were observed at 10 μg/mL FRIL with unpurified Influenza virus using negative staining EM (FIG. 15A). Influenza particles were bound at much closer proximity to one another by FRIL compared to untreated virus particles, with visible FRIL proteins surrounding the viral aggregates. Large and densely packed clumps of overlapping influenza particles and FRIL protein were observed at 150 μg/mL FRIL concentrations but not in untreated virus particles (FIG. 15A).

To better observe the aggregation of Influenza particles, we then conducted the protocol with sucrose-gradient purified viruses and 150 μg/mL purified FRIL. Aggregates of Influenza virus particles can be observed surrounding FRIL aggregates (FIG. 15B). The amount of isolated Influenza virus, which can be observed abundantly in untreated Influenza virus, is greatly decreased with the addition of FRIL.

These images suggest that FRIL's carbohydrate-binding domain (CBD) on each of its four monomers could connect multiple virus particles, thereby entrapping virions in large aggregates at 10-150 μg/mL of FRIL protein (FIG. 15). The clustering of virus particles could explain FRIL's debulking mechanisms: the entrapped Influenza virus particles would be neutralized and removed from the sample during centrifugation. Furthermore, other virus-trapping lectins MBL and SP-D have been documented to promote viral clearance with the immune system [82], and FRIL could have a similar effect. Since the spike (S) protein present on the SARS-CoV-2 virus envelope encodes 22 complex-type N-linked glycan sites per monomer [41], we hypothesize that FRIL could entrap and debulk SARS-CoV-2 through binding to the N-glycan sites on spike protein in the microbubble assay. Mainly, when the patient sample was incubated with lablab bean powder, FRIL protein released from the lablab bean powder could entrap SARS-CoV-2 virus particles to form into clumps. The entrapped virus particles are then removed during the centrifugation, leading to a lower microbubble count.

### Advancing Chewing Gum Delivery of Viral Trap Proteins from Laboratory to the Clinic

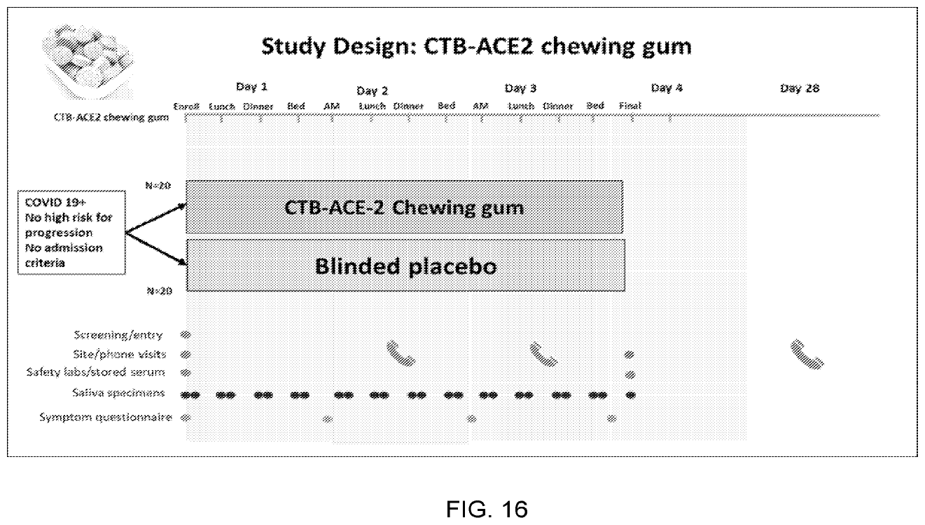

While ex vivo SARS-CoV-2 debulking is effective for various SARS-CoV-2 strains, including the highly transmissible omicron strain, the time required for repopulation of saliva by SARS-CoV-2 is still unknown. While current CTB-ACE2 gum could be used for short duration (in dental clinics, public transportation, gatherings, restaurants etc.), therapeutic applications of the CTB-ACE2 gum for successful reduction of viral load in COVID-19 patients would require data on viral load kinetics in saliva. We will be conducting a phase I/II placebo-controlled, double-blind study of CTB-ACE2 chewing gum as detailed in the study plan shown in FIG. 16.

The primary safety analyses will be conducted on all participants receiving study product and presented by age strata, all solicited and unsolicited AEs will be summarized by frequency per arm for the whole group, and stratified by age, as percentages, along with associated exact 95% Clopper-Pearson confidence intervals. For the virology endpoints, levels of SARS-CoV-2 RNA on days 1.2.3.4 will be compared between arms using non-parametric Wilcoxon rank-sum tests and descriptive statistics, separately at each scheduled measurement time. In addition, viral antigens (N or spike protein) will be quantified in saliva samples using microbubble or RAPID assays shown above. For the evaluation of the clinical evolution of COVID 19, the severity ranking will be based on the area under the curve AUC of the daily total symptom score associated with COVID-19 over time.

### CONCLUSION

Approximately one fourth of American population chew gum 2-3 times a week mostly for pleasure, although delivery of small molecules is used currently or in the past for delivering aspirin, nicotine, caffeine or for enhancing oral hygiene or health. Attempts to deliver therapeutic proteins like insulin using chewing gum have failed so far, primarily because of degradation in the digestive system. Our data show that delivery of neutralizing pathogens via chewing gum to the oral cavity or in the throat surface is effective to debulk viral load and reduce transmission of infection. Most importantly, proteins produced in plants have shown incredible stability in the gum during production at high temperature and long-term storage at ambient temperature. This study illustrates the power of delivering viral trap proteins in chewing gum to reduce infection and transmission of SARS-CoV-2 or influenza viruses and the potential to advance this platform to various other orally transmitted viral, bacterial or fungal diseases.

### REFERENCES

1. Bowen, W. H., Burne, R. A., Wu, H., Koo, H. (2018) Oral biofilms: pathogens, matrix, and polymicrobial interactions in microenvironments. Trends Microbiol. 26, 229-242.
2. Huang, N., Pérez. P., Kato, T., Mikami, Y., Okuda, K., Gilmore, R. C., Dominguez Conde, C., Gasmi, B., Stein, S., Beach, M et al. (2021) SARS-CoV-2 infection of the oral cavity and saliva. Nature Medicine. 27, 892-903 (DOI:10.1038/s41591-021-01296-8).
3. Li, Y., Ren, B., Peng, X., Hu, T., Li, J., Cong. T., Tang, B., Xu, X., Zhou, X. (2020) Saliva is a non-negligible factor in the spread of COVID-19. Mol Oral Microbiol. 35, 141-145 (DOI:10.1111/omi.12289).
4. Chan, J. F-W., Yip. C. C., To, K. K., Tang, T. H., Wong, S. C., Leung, K., Fung, A. Y., Ng, A. C., Zou, Z., Tsoi, H., et al. (2020) Improved Molecular Diagnosis of COVID-19 by the Novel, Highly Sensitive and Specific COVID-19-RdRp/Hel Real-Time Reverse Transcription-PCR Assay Validated In Vitro and with Clinical Specimens. J Clin Microbiol. 58, 10-20 (DOI:10.1128/JCM.00310-20).
5. Wölfel, R., Corman, V. M., Guggemos, W., Seilmaier, M., Zange, S., Muller, M. A., Niemeyer, D., Jones, T. C., Vollmar, P., Rothe, C. et al. (2020) Virological assessment of hospitalized patients with COVID-2019. Nature. 581, 465-469 (DOI:10.1038/s41586-020-2196-x).
6. Atyeo, N., Rodriguez, M. D., Papp, B. & Toth. Z. (2021) Clinical Manifestations and Epigenetic Regulation of Oral Herpesvirus Infections. Viruses. 13(681), 2-17 (DOI: 10.3390/v13040681).
7. Tewari, P., Banka, P., Kernan. N., Reynolds, S., White, C., Pilkington, L., O'Toole, S., Sharp, L., D'Arcy, T., Cliona, M. et al. (2021) Prevalence and concordance of oral HPV infections with cervical HPV infections in women referred to colposcopy with abnormal cytology. J Oral Pathol Med. 50(7), 692-699. (DOI:10.1111/jop.13172).
8. Paules, C. & Subbarao, K. (2017) Influenza. The Lancet. 390, 697-708. (DOI:10.1016/S0140-6736(17)30129-0).
9. Jang, Y. H. & Seong. B. L. (2019) The Quest for a Truly Universal Influenza Vaccine. Front. Cell. Infect. Microbiol. 9, 344. (DOI:10.3389/fcimb.2019.00344).
10. Liu, W. J., Xiao, H., Dai, L., Liu, D., Chen, J., Qi, X., Bi, Y., Shi, Y., Gao, G. F., Liu. Y. (2021) Avian influenza A (H7N9) virus: from low pathogenic to highly pathogenic. Front. Med. 15(4), 507-527 (DOI:10.1007/s11684-020-0814-5).
11. Wu, Y., Wu, Y., Tefsen, B., Shi, Y. & Gao, G. F. (2014) Bat-derived influenza-like viruses H17N10 and H18N11. Trends in Microbiology. 22, 183-191 (DOI:10.1016/j.tim.2014.01.010).
12. Singanayagam, A., Hakki, S., Dunning, J., Madon, K. J., Crone, M. A., Koycheva. A., Derqui-Fernandez, N., Barnett, J. L., Whitfield. M. G., Varro, R. et al. (2022) Community transmission and viral load kinetics of the SARS-CoV-2 delta (B.1.617.2) variant in vaccinated and unvaccinated individuals in the U.K.: a prospective, longitudinal, cohort study. Lancet Infect Dis. 22(2), 183-195.
13. Wang R, Chen J, Wei G-W (2021) Mechanisms of SARS-CoV-2 evolution revealing vaccine resistant mutations in Europe and America. J. Phys. Chem. Lett. 12, 11850-11857 (https://doi.org/10.1021/acs.jpclett.1c03380).
14. Stadnytskyi V, Anfinrud P, Bax A (2021) Breathing, speaking, coughing or sneezing: what drives transmission of SARS-CoV-2? Journal of Internal Medicine. 290, 1010-1027 (https://onlinelibrary/wiley.com/doi/full/10.1111/joim.133260.10076/s00784-020-03413-2).
15. Anfinrud, P., Stadnytskyi, V., Bax, C. E., Bax, A. (2020) Visualizing Speech-Generated Oral Fluid Droplets with Laser Light Scattering. N. Engl. J. Med. 382, 2061-2063.
16. Bax, A., Bax, C. E., Stadnytskyi. V., Anfinrud, P. (2021) SARS-CoV-2 transmission via speech-generated respiratory droplets. The Lancet Infectious diseases. 21(3), 318.
17. Bahl, P., de Silva, C., Bhattacharjee, S., Stone, H., Doolan, C., Chughtai, A. A., Raina Macintyre, C. R. (2020) Droplets and Aerosols generated by singing and the risk of COVID-19 for choirs. Clin Infect Dis 72, 639-641.
18. Alsved, M., Matamis, A., Bohlin, R., Richter, M., Bentsson, P. E., Fraenkel, C.-J., Medstrand, P., Löndahl, J. (2020) Exhaled respiratory particles during singing and talking. Aerosol Sci. Technol. 54, 1245-1248.
19. Smith, S. H., Somsen, G. A., Rijn, C. V., Kooij, S., van der Hoek, L., Bem, R. A., Bonn, D. (2020) Aerosol persistence in relation to possible transmission of SARS-CoV-2. Phys. Fluids. 32:107108 (DOI: 10.1063/5.0027844).
20. Stadnytskyi, V., Bax. C. E., Bax. A., Anfinrud, P. (2020) The airborne lifetime of small speech droplets and their potential importance in SARS-CoV-2 transmission. Proc. Natl. Acad. Sci. USA. 117(22), 11875-11877.
21. Gregson, F. K. A., Watson, N. A., Orton, C. M., Haddrell, A. E., McCarthy, L. P., Finnie, T. J. R. Gent, N., Donaldson, G. C., Shah, P. L., Calder, J. D. (2021) Comparing aerosol concentrations and particle size distributions generated by singing, speaking and breathing. Aerosol Sci. Technol. 55(6), 681-691.
22. Lam-Hine, T., McCurdy. S. A., Santora, L., Duncan, L., Corbett-Detig, R., Kapusinszky, B., Willis, M. et al., (2021) Outbreak Associated with SARS-CoV-2 B.1.617.2 (Delta) Variant in an Elementary School-Marin County, California, May-June 2021. MMWR-Morb Mortal Wkly Rep. 70(35), 1214-1219.
23. CDC (2021) Scientific Brief: SARS-CoV-2 Transmission. (https://www.cdc.gov/coronavirus/2019-ncov/science/science-briefs/sars-cov-2-transmission.html).
24. Asadi, S., Wexler, A. S., Cappa, C. D., Barreda, S., Bouvier, N. M., Ristenpart, W. D. (2020) Effect of voicing and articulation manner on aerosol particle emission during human speech. PLOS One. 15(1): e0227699 (https://doi.org/10.1371/journal.pone.0227699).
25. Majra, D., Benson, J., Pitts, J., Stebbing, J. (2021) SARS-CoV-2 (COVID-19) superspreader events. J. Infection. 82(1), 36-40.
26. Asadi. S., Wexler, A. S., Cappa, C. D., Barreda, S., Bouvier, N. M., Ristenpart, W. D. (2019) Aerosol emission and superemission during human speech increase with voice loudness. Sci Rep 9: 2348 (http://doi.org/10.1038/s41598-019-38808-z).
27. Chao, C. Y. H., Wan, M. P., Morawska, L., Johnson, G. R., Ristovski, Z. D., Hargreaves, M., Mengersen, K., Corbett, S., Li, Y., Xie, X., Katoshevski, D. (2009) Characterization of expiration air jets and droplet size distributions immediately at the mouth opening. J. Aerosol Sci. 40(2), 122-133.
28. Kushalnagar, P., Chow, C. C., Bax, A. (2021) Self-infection with speech aerosol may contribute to COVID-19 severity. J Intern Med. 290(6), 1275-1277.
29. Ferrer, M. D., Barrueco, A. S., Martinez-Beneyto, Y., Mateos-Moreno, M. V., Ausina-Marquez, V., Garcia-Vázquez, E., Puche-Torres, M., Forner Giner, M. J., González, A. C., Coello, J. M. S, et al. (2021) Clinical evaluation of antiseptic mouth rinses to reduce salivary load of SARS-CoV-2. Scientific Reports. 11:24392 (https://doi.org/10.1038/s41598-021-03461-y).
30. Xu, Y., Li, X., Zhu, B., Liang, H., Fanf, C., Gong, Y., Guo, Q., Sun, X., Zhao, D., Shen, J., Zhang, H, Liu, H et al. (2020) Characteristics of pediatric SARS-CoV-2 infection and potential evidence for persistent fecal viral shedding. Nat. Med. 26(4), 502-505.
31. Herrera, D., Serrano, J., Roldan, S. & Sanz, M. (2020) Is the oral cavity relevant in SARS-CoV-2 pandemic? Clin. Oral Investig. 24(8), 2925-2930.
32. Katz. M. H. (2021) Challenges in Testing for SARS-CoV-2 Among Patients Who recovered From COVID-19. JAMA Intern Med. 181(5), 704-705.
33. Daniell, H., Mangu, V., Yakubov, B., Park, J., Habibi, P., Shi, Y., Gonnella, P. A., Fisher, A., Cook. T., Zeng. L et al. (2020) Investigational new drug enabling angiotensin oral-delivery studies to attenuate pulmonary hypertension. Biomaterials. 233:119750 (DOI:10.1016/j.biomaterials.2019.119750).
34. Daniell, H., Nair, S. K., Esmacili, N., Wakade, G., Shahid, N., Ganesan, P. K., Islam, Md.R., Shepley-Mctaggart, A., Feng, S., Gary, E. N. et al. (2021) Debulking SARS-CoV-2 in saliva using angiotensin converting enzyme 2 in chewing gum to decrease oral virus transmission and infection. Molecular Therapy. (DOI: 10.1016/j.ymthe.2021.11.008).
35. Bracken, C. J., Lim, S. A., Solomon, P., Rettko, N. J. Nguyen, D. P., Zha, B. S., Schaefer, K., Byrnes, J. R., Zhou, J., Lui, I., Liu, J., Pance, K., QCRG Structural Biology Consortium, Zhou, X. X., Leung, K. K, & Wells, J. A. (2021) Bi-paratopic and multivalent VH domains block ACE2 binding and neutralize SARS-CoV 2. Nat Chem Biol. 17, 113-121 (DOI:10.1038/s41589-020-00679-1).
36. Glasgowa, A., Glasgowb, J., Limontac, D., Solomonb, P., Luib, I., Zhang, Y., Nixe, M. A., Rettkob, N. J., Zhaf, S., Yaming, R., Kao, K., Rosenberg, O. S., Ravetchg. J. V., Wiita, A. P., Leung, K. K., Limb, S. A., Zhou, X. X., Hobman, T. C., Kortemme, T. & Wells, J. A. (2020) Engineered ACE2 receptor traps potently neutralize SARS-CoV-2. Proc Natl Acad Sci USA. 117, 28046-28055 (DOI:10.1073/pnas.2016093117).
37. Lv J. Gao J. Wu B, Yao M, Yang Y, Chai T and Li N. (2021) Acrosol Transmission of Coronavirus and Influenza Virus of Animal Origin. Front. Vet. Sci. 8:572012 (DOI: 10.3389/fvets.2021.572012).
38. Liu, Y., Shahed-Al-Mahmud, M., Chen, X., Chen, T., Liao. K., Lo, J. M., Wu. Y., Ho, M., Wu. C., Wong. C., Jan. J., Ma, C. (2020) Cell Rep. 32(6), 108016-108020.
39. Bagdonaite, I., Wandall, H. H. (2018) Global aspects of viral glycosylation. Glycobiol. 28, 443-467.
40. Krammer, F., Smith, G. J. D., Fouchier, R. A. M., Peiris, M., Kedzierska, K., Doherty, P. C., Palese, P., Shaw, M. L., Treanor, J., Webster, R. G., Garcia-Sastre, A. (2018) Influenza. Nat Rev Dis Primers. 4(1), 3 (DOI: 10.1038/s41572-018-0002-y).
41. Watanabe, Y., Allen, J. D., Wrapp, D., Mclellan, J. S., Crispin, M. (2020) Site-specific glycan analysis of the SARS-CoV-2 spike. Science. 369(6501). 330-333.
42. Taubenberger, J. K., Moren, D. M. (2008) The Pathology of Influenza Virus infections. Annu Rev Pathol. 3, 499-522.
43. Hsich, Y. H., Wu, J., Fang, J., Yang. Y., Lou, J. (2014) Quantification of bird-to-bird and bird-to-human infections during 2013 novel H7N9 avian influenza outbreak in China. PLOS One. 9(2): e111834. DOI: 10.1371/journal.pone.0111834.eCollection 2014.
44. Shenoy, V., Kwon K. C., Rathinasabapathy, A., Lin, S., Jin. G., Song. C., Shil, P., Nair, A., Qi, Y., Li, Q., et al. (2014) Oral Delivery of Angiotensin-Converting Enzyme 2 and Angiotensin-(1-7) Bioencapsulated in Plant Cells Attenuates Pulmonary Hypertension. Hypertension. 64, 1248-1259.
45. Shil, P., Kwon, K. C., Zhu, P., Verma, A., Daniell, H., Li, Q. (2014) Oral Delivery of ACE2/Ang-(1-7) Bioencapsulated in Plant Cells Protects against Experimental Uveitis and Autoimmune Uveoretinitis. Molecular Therapy. 22, 2069-2082.
46. Khan I, Daniell H. (2021) Oral delivery of therapeutic proteins bioencapsulated in plant cells: preclinical and clinical advances. Current Opinion in Colloid and Interface Science 54: 101452.
47. Park, J., Yan, G., Kwon, K. C., Liu, M., Gonnella, P. A., Yang, S., and Daniell, H. (2020) Oral delivery of novel human IGF-1 bioencapsulated in lettuce cells promotes musculoskeletal cell proliferation, differentiation, and diabetic fracture healing. Biomaterials. 233: 119591 (DOI: 10.1016/j.biomaterials.2019.119591).
48. He. W., Baysal, C., Lobato Gómez, M., Huang. X., Alvarez, D., Zhu, C., ArmarioNajera, V., Blanco Perera, A., Cerda Bennaser, P., Saba-Mayoral, A., et al. (2021) Contributions of international plant science community to fight against infectious diseases in human—part 2: affordable drugs in edible plants for endemic and reemerging diseases. Plant Biotechnol. J. 19, 1921-1936.
49. Kwon, C., Daniell, H. (2016). Oral Delivery of Protein drugs Bioencapsulated in Plant Cells. Mol Ther. 24(8), 1342-1350.
50. Singh R, Ren Z, Shi Y. Lin S, Kwon K C, Balamurugan S, Rai V, Mante F, Koo H, Daniell H. (2021) Affordable oral health care: dental biofilm disruption using chloroplast made enzymes with chewing gum delivery. Plant Biotechnol J. 19(10), 2113-2125 (DOI: 10.1111/pbi.13643).
51. Daniell, H., Kulis, M. & Herzog, R. W. (2019) Plant cell-made protein antigens for induction of Oral tolerance. Biotechnology Advances. 37: 107413 (DOI:10.1016/j.biotechadv.2019.06.012).
52. Daniell, H., Jin, S., Zhu, X., Gitzendanner, M. A., Soltis, D. E., Soltis, P. S. (2021) Green giant—a tiny chloroplast genome with mighty power to produce high-value proteins: history and phylogeny. Plant Biotechnol J. 19, 430-447 (DOI:10.1111/pbi.13556).
53. Srinivasan, A., Herzog, R. W., Khan, I., Sherman, A., Bertolini. T., Wynn, T., Daniell, H. et al. (2021) Preclinical development of plant-based oral immune modulatory therapy for hemophilia B. Plant Biotechnol J. 19(10), 1952-1966. (DOI:10.1111/pbi.13608).
54. Kaushik. P., Kaushik, D. (2019) Medicated Chewing Gums: Recent Patents and Patented technology Platforms. Recent Put Drug Deliv Formul. 13(3), 184-191.
55. Greene, T., Rogers, S., Franzen, A., Gentry. R. (2017) A critical review of the literature to conduct a toxicity assessment for oral exposure to methyl salicylate. Critical Reviews In Toxicology. 47(2), 98-120.
56. Ryan, E J., Kim. C., Fickes, E. J., Williamson, M., Muller, M. D., Barkley, J. E., Gunstad, J., Glickman, E. L. (2013) Caffeine gum and cycling performance: a timing study. J Strength Cond Res. 27(1), 259-64.
57. Brown, R., Sam, C., Green, T., Wood, S. Effect of GutsyGum™. (2015) A Novel Gum, on Subjective Ratings of Gatsro Esophageal Reflux Following A Refluxogenic Meal. J Diet Suppl. 12(2), 138-45.
58. Simons, D., Brailsford, S., Kidd E A., Beighton D (2001) The effect of chlorhexidine acetate/xylitol chewing gum on the plaque ad gingival indices of elderly occupants in residential homes. J Clin Periodontol. 28(11), 1010-5.
59. Wennike, P., Danielsson, T., Landfeldt, B., Westin, A., Tonnesen, P. (2003) Smoking reduction promotes smoking cessation: results from a double blind, randomized, placebo-controlled trial of nicotine gum with 2-year follow-up. Addiction. 98(10), 1395-402.
60. Round, E K., Chen, P., Taylor A K., Schmidt, E. (2019) Biomarkers of Tobacco Exposure Decrease After Smokers Switch to an E-Cigarette or Nicotine Gum. Nicotine Tob Res. 21(9), 1239-1247.
61. Söderling. E., Pienihäkkinen K. (2022) Effects of xylitol chewing gum and candies on the accumulation of dental plaque: a systematic review. Clinical Oral Investigations. 26, 119-129.
62. Freitas, A. A. R., Ribeiro, A J., Santos. A C., Veiga, F., Nunes, L C. C., Silva, D A., Soares-Sobrinho, J. L., Silva-Filho, E. C. (2020). Sterculia striata gum as a potential oral delivery system for protein drugs. International Journal of Biological Macromolecules. 164, 1683-1692.
63. Boyhan, D., Daniell, H. (2011). Low-cost production of proinsulin in tobacco and lettuce chloroplasts for injectable or oral delivery of functional insulin and C-peptide. Plant Biotechnol J. 9(5), 585-98.
64. Everett. J., Hokama. P., Roche. A M., Reddy. S., Hwang. Y., Kessller. L., Glaascock. A., Li. Y., Whelan. J N., Weiss. S R. (2021) SARS-CoV-2 Genomic Variation in Space and Time in Hospitalized Patients in Philadelphia. mBio. 12(1), e03456-20 (DOI: 10.1128/mBio.03456-20).
65. Marques. A D., Sherrill-Mix. S., Everett. J K., Reddy. S., Hokama. P., Roche. A M., Hwang. Y., Glascock. A., Whiteside. S A., Graham-Wooten. G. (2022) SARS-CoV-2 Variants Associated with Vaccine Breakthrough in the Delaware Valley through Summer 2021. mBio. 13(1), e03788-21 (DOI: 10.1128/mbio.03788-21).
66. Smith. B F., Graven. P F., Yang. D Y., Downs. S M., Hansel. D E., Faan. G., Qin. X. (2022) Using Spike Gene Target Failure to Estimate Growth Rate of the Alpha and Omicron Variants of SARS-CoV-2. J Clin Microbiol. 60(4): e0257321
67. Brown K A, Gubbay J, Hopkins J., Patel, S., Buchan, S. A A., Daneman, N., Goneau, L. W. (2021) S-Gene Target Failure as a Marker of Variant B.1.1.7 Among SARS-CoV-2 Isolates in the Greater Toronto Area, December 2020 to March 2021. JAMA, 325(20):2115-2116 (DOI:10.1001/jama.2021.5607).
68. Chen, H., Li, Z., Zhang, L., Sawaya, P., Shi, J., and Wang, P. (2019) Quantitation of femtomolar-level protein biomarkers using a simple microbubbling digital assay and bright-field smartphone imaging. Angew. Chem. Int. Ed. 58(39), 13922-13928.
69. Chen, H., Li, Z., Feng, S., Richard-Greenblatt, M., Hutson, E., Andrianus, S., Glaser, L J., Rodino, KG., Qian, J., Jayaraman, D., Collman, R G., Glascock, A., Bushman, FD., Lee, J S., Cherry, S., Fausto, A., Weiss, S R., Koo, H., Corby, P M., O'Doherty. U., Garfall, A L., Vogl, D T., Stadtmauer, E A., Wang, P. (2022) Femtomolar SARS-CoV-2 Antigen Detection Using the Microbubbling Digital Assay with Smartphone Readout Enables Antigen Burden Quantitation and Tracking. Clin Chem. 68 (1), 230-239.
70. Torres, M. D. T., de Araujo, W. R., de Lima, L. F., Ferreira, A. L., de la Fuente-Nunez, C. (2021) Low-Cost Biosensor for Rapid Detection of SARS-CoV-2 at the Point of Care. Matter. 4 (7), 2403-2416 (https://doi.org/10.1016/j.matt.2021.05.003).
71. Torres, M. D. T., de Lima, L. F., Ferreira, A. L., de Araujo, W. R., Callahan, P., Dávila, A., Abella, B. S., de la Fuente-Nunez, C. (2022) Detection of SARS-CoV-2 with RAPID: A Prospective Cohort Study. iScience. 25 (4): 104055 (https://doi.org/10.1016/j.isci.2022.104055).
72. Yang, J., Petitjean, S. J. L., Koehler, M., Zhang. Q., Dumitru, A. C., Chen, W., Derclaye, S.; Vincent, S. P., Soumillion, P., Alsteens. D. (2020) Molecular Interaction and Inhibition of SARS-CoV-2 Binding to the ACE2 Receptor. Nat. Commun. 11:4541 (https://doi.org/10.1038/s41467-020-18319-6).
73. Andersen, K. G., Rambaut, A., Lipkin, W. I., Holmes, E. C., Garry, R. F. (2020) The Proximal Origin of SARS-CoV-2. Nat. Med. 26 (4), 450-452. (https://doi.org/10.1038/s41591-020-0820-9).
74. Muñoz, J., Montes, R., Baeza, M. (2017) Trends in Electrochemical Impedance Spectroscopy Involving Nanocomposite Transducers: Characterization, Architecture Surface and Bio-Sensing. TrAC Trends Anal. Chem. 97, 201-215. (https://doi.org/10.1016/j.trac.2017.08.012).
75. Pereira, A. R., Sedenho, G. C., Souza, J. C. P. DE., Crespilho, F. N. (2018) Advances in Enzyme Bioelectrochemistry. An. Acad. Bras. Cienc. 90 (1 suppl 1), 825-857. (https://doi.org/10.1590/0001-3765201820170514).
76. Mauritz, K. A., Moore, R. B. (2004) State of Understanding of Nafion. Chem. Rev. 104 (10), 4535-4586 (https://doi.org/10.1021/cr0207123).
77. Sco, G., Lec, G., Kim, M. J., Back, S.-H., Choi, M., Ku, K. B., Lec, C.-S., Jun, S., Park, D., Kim, H. G., et al. (2020) Rapid Detection of COVID-19 Causative Virus (SARS-CoV-2) in Human Nasopharyngeal Swab Specimens Using Field-Effect Transistor-Based Biosensor. ACS Nano. 14 (4), 5135-5142 (https://doi.org/10.1021/acsnano.0c02823).
78. Rashed, M. Z., Kopechek, J. A., Priddy, M. C., Hamorsky, K. T., Palmer, K. E., Mittal, N., Valdez, J., Flynn, J., Williams, S. J. (2021) Rapid Detection of SARS-CoV-2 Antibodies Using Electrochemical Impedance-Based Detector. Biosens. Bioelectron. 171: 112709 (https://doi.org/10.1016/j.bios.2020.11).
79. Li, Y., Liu, D., Wang, Y., Su, W., Liu, G., & Dong, W. (2021) The Importance of Glycans of Viral and Host Proteins in Enveloped Virus Infection. Frontiers in immunology. 12, 1-12 (https://doi.org/10.3389/fimmu.2021.63873).
80. Luo, S., Hu, K., He, S., Wang. P., Zhang. M., Huang, X., Du. T., Zheng. C., Liu, Y., Hu. Q. (2015) Contribution of N-linked glycans on HSV-2 gB to cell-cell fusion and viral entry. Virology. 483, 72-82 (DOI:10.1016/j.virol.2015.04.005).
81. Lagerlöf, F., & Dawes, C. (1984) The volume of saliva in the mouth before and after swallowing. Journal of dental research. 63: 618-621.
82. Hartshorn, K. L., White, M. R., Shepherd, V., Reid, K., Jensenius, J. C., & Crouch, E. C. (1997). Mechanisms of anti-influenza activity of surfactant proteins A and D: comparison with serum collections. The American journal of physiology. 273(6), 1156-1166 (https://doi.org/10.1152/ajplung.1997.273.6.L1156).
83. Letting, F. K., Venkataramana. P. B., Ndakidemi, P. A. (2021) Breeding potential of lablab (Lablab purpureus): a review on characterization and bruchid studies towards improved production and utilization in Africa. Genet Resour Crop Evol. 68. 3081-3101.
84. Hossain. S., Ahmed, R., Bhowmick, S., Sarkar, M., Nahar, T., Uddin, B., Basunia, M., Mamun, A., Hashimoto, M., Shido, O. (2020) Lallab purpureus bean flour ameliorates plasma proteins and accretion of docosahexaenoic acid in the plasma, liver and brain of malnourished rats. Legume Science. 1-13 (https://doi.org/10.1002/leg3.34).
85. Martyn, D. M., Lau, A. (2019). Chewing gum consumption in the United States among children, adolescents and adults. Food Additives & Contaminants:Part A. 36(3), 350-358.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for debulking viral load from the oral cavity in a subject, the method comprising orally administering to the subject a therapeutically effective amount of a composition comprising a carrier including a trapping molecule having affinity for a protein or glucan on the surface of a virus, said trapping molecule binding said surface protein or glucan thereby trapping said virus within said carrier and reducing viral load in said oral cavity of said subject.

2. The method of claim 1, wherein the administering takes place before or after the subject is exposed to the virus and reduces recovery time for, eliminates, or minimizes at least one complication from viral infection.

3. The method of claim 1, further comprising reducing bacterial and, or fungal load in the oral cavity, wherein said composition comprises a trapping molecule having affinity for the surface of said bacteria and, or fungus.

4. The method of claim 3, wherein said trapping molecule is selected from one or more of ACE2, CTB-ACE2, an IVA blocking peptide, FRIL, an antimicrobial peptide, and lipase, and said carrier is a chewing gum, long-acting lozenge, or a tablet.

5. The method of claim 1, wherein said virus is selected from corona virus, influenza virus, herpes virus, cytomegalovirus, papilloma virus, Epstein Barr virus, hepatitis virus, zika virus, and HHV-7.

6. The method of claim 1, wherein the virus comprises a Coronavirus selected from an Alphacoronavirus, a Betacoronavirus, a Gammacoronavirus, a Deltacoronavirus, MERS-CoV, SARS-CoV, and SARS-CoV-2.

7.-9. (canceled)

10. The method of claim 1 wherein said virus is SARS-CoV-2 and said trapping molecule is ACE2, CTB-ACE2, an IVA blocking peptide, FRIL which traps a spike protein containing virus and, or said virus is an influenza virus A (IVA) virus and said trapping molecule is a IVA blocking peptide or FRIL, which traps influenza viral particles in said carrier, thereby reducing viral load in the oral cavity.

11. The method of claim 5, wherein the virus is an Alpha influenza virus and comprises at least one of Influenza A virus, Influenza B virus, and Influenza C virus.

12. The method of claim 11, wherein said trapping molecule is an influenza virus A (IVA) blocking peptide comprising virus binding portions of HA and, or neuraminidase proteins and, or a FRIL molecule that binds a glucan on a virus surface, and traps influenza viral particles in said carrier, said carrier being selected from selected from a chewing gum, long-acting lozenge, or a tablet.

13.-21. (canceled)

22. A composition comprising an effective amount of a trapping molecule having binding affinity for a molecule on a virus surface, said virus being optionally transmissible via aerosolization and said trapping molecule being present in a carrier suitable for oral administration, wherein said virus is selected from one or more of a Corona Virus, an Influenza A virus, Herpes virus or Papilloma virus, said trapping molecule is selected from one or more of ACE2, CTB-ACE2, an IVA blocking peptide, and a FRIL protein from lablab bean powder which binds a glucan on the surface of said virus and said carrier is chewing gum.

23. The composition of claim 22, wherein said chewing gum includes a gum base comprising (28.2%), maltitol (20.4%), sorbitol (13%), xylitol (13%), isomalt (13%), natural and artificial flavors, magnesium stearate (3%), silicon dioxide (0.43%), stevia (0.65%).

24. A method for debulking pathogenic microorganism load and, or virus load from the oral cavity in a subject, the method comprising orally administering to the subject a therapeutically effective amount of a composition comprising a carrier including a trapping molecule having affinity for said microorganism and, or virus, wherein binding of said microorganism and or virus to said trapping molecule traps said microorganism and, or virus within said carrier and thereby reducing microorganism and or virus load in said oral cavity of said subject.

25. The method of claim 24, wherein the microorganism is a bacteria and, or a fungus and the administering takes place before or after the subject is exposed to the bacteria wherein the administering reduces recovery time for, eliminates, or minimizes at least one complication from bacterial and, or fungal infection.

26.-27. (canceled)

28. The method of claim 24 wherein said bacteria is Streptococcus pyrogenes which causes strep throat, said fungus is C. albicans and said virus is a corona virus or an influenza virus.

29. (canceled)

30. The composition of claim 28, wherein said trapping molecule is one or more of an anti-microbial peptide, lipase or ACE2-CTB and said carrier is a chewing gum, long-acting lozenge, or a tablet.

31. The composition of claim 28, wherein said bacteria is S. pyrogenes, said trapping molecule is an antimicrobial peptide and said carrier is chewing gum.

32. (canceled)

33. The composition of claim 25, wherein said fungus is C. albicans, said trapping molecule is lipase and said carrier is chewing gum.
